(19) United States
(12) Patent Application Publication
(10) Pub. No.: US 2017/0106067 A1
(43) Pub. Date: Apr. 20, 2017

(54) COMBINATORIAL IMMUNOTHERAPY FOR PANCREATIC CANCER TREATMENT

(71) Applicant: THE JOHNS HOPKINS UNIVERSITY, Baltimore, MD (US)

(72) Inventors: Elizabeth Jaffee, Lutherville, MD (US); Lei Zheng, Ellicott City, MD (US)

(21) Appl. No.: 15/317,425

(22) PCT Filed: Jun. 12, 2015
(86) PCT No.: PCT/US2015/035646

§ 371 Date: Dec. 9, 2016

Related U.S. Application Data
(60) Provisional application No. 62/011,101, filed on Jun. 12, 2014.

(51) Int. Cl. A61K 39/00; A61K 39/395; C07K 16/28; A61K 31/675

(52) U.S. Cl. CPC A61K 39/0011; A61K 31/675; A61K 39/3955; C07K 16/2803; C07K 16/2827; A61K 2039/5152; A61K 2039/523; A61K 2039/522; A61K 2039/505; C07K 2317/73; C07K 2317/76; A61K 2039/5156; A61K 2039/54

(57) ABSTRACT

The invention features compositions and methods for treating and preventing pancreatic cancer.

### RELATED APPLICATIONS

This application claims the benefit of priority under 35 U.S.C. §119(e) to U.S. Provisional Application No: 62/011,101, filed Jun. 12, 2014, which is incorporated herein by reference in its entirety.

### STATEMENT OF RIGHTS TO INVENTIONS MADE UNDER FEDERALLY SPONSORED RESEARCH

This invention was made with government support under grant number K23 CA148964-01 awarded by The National Institutes of Health, under grant number T32 DK 7713-18 awarded by The National Institute of Diabetes and Digestive and Kidney Diseases (NIDDK), and under grant number P50 CA062924 awarded by The National Cancer Institute Specialized Programs of Research Excellence (SPORE) in Gastrointestinal Cancers. The government has certain rights in the invention.

### FIELD OF THE INVENTION

This invention relates generally to the field of oncology.

### BACKGROUND OF THE INVENTION

Prior to the invention described herein, pancreatic ductal adenocarcinoma (PDA) had a poor prognosis due to late detection and resistance to conventional therapies. As such, there is a pressing need to identify additional treatment options for PDA.

### SUMMARY OF THE INVENTION

The invention is based, at least in part, on the surprising discovery that programmed death 1 (PD-1)/ PD-1 ligand (PD-L1) blockade together with vaccine therapy facilitates effector T cell infiltration into pancreatic tumors. Specifically, as described in detail below, treatment with a vaccine-based immunotherapy (e.g., a granulocyte-macrophage colony-stimulating factor (GM-CSF) vaccine (GVAX) or a Listeria monocytogenes (Lm)-based vaccine that expresses an Annexin A2 (ANXA2) antigen) in pancreatic cancer induces adaptive resistance with induction of PD-L1 expression. As result, the pancreatic cancer tumor microenvironment selectively utilizes the PD-1/PD-L1 pathway to mediate vaccine induced immune suppression. However, as described herein, this immune suppression was overcome by combining vaccine therapy with PD-1/PD-L1 blockade treatment resulting in prolonged survival and improved cure rates of pancreatic cancer bearing mice.

Provided herein are methods of treating or preventing cancer in a subject comprising administering a vaccine to the subject and administering a PD-1 inhibitor or a PD-L1 inhibitor to the subject, thereby treating or preventing the cancer in the subject. Preferably, the methods described herein inhibit the growth or progression of cancer, e.g., a tumor, in a subject. For example, the methods described herein inhibit the growth of a tumor by at least 1%, e.g., by at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, at least 90% or 100%. In other cases, the methods described herein reduce the size of a tumor by at least 1 mm in diameter, e.g., by at least 2 mm in diameter, by at least 3 mm in diameter, by at least 4 mm in diameter, by at least 5 mm in diameter, by at least 6 mm in diameter, by at least 7 mm in diameter, by at least 8 mm in diameter, by at least 9 mm in diameter, by at least 10 mm in diameter, by at least 11 mm in diameter, by at least 12 mm in diameter, by a least 13 mm in diameter, by at least 14 mm in diameter, by at least 15 mm in diameter, by at least 20 mm in diameter, by at least 25 mm in diameter, by at least 30 mm in diameter, by at least 40 mm in diameter, by at least 50 mm in diameter or more.

In some cases, the cancer comprises a gastrointestinal cancer, e.g., pancreatic cancer, e.g., pancreatic ductal adenocarcinoma (PDA).

The subject is preferably a mammal in need of such treatment, e.g., a subject that has been diagnosed with cancer, e.g., pancreatic cancer, or a predisposition thereto, i.e., at risk of developing pancreatic cancer. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Modes of administration include intravenous, systemic, oral, rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy is also provided.

Any vaccine for the treatment of cancer, e.g., pancreatic cancer, e.g., PDA, is useful in the methods described herein.

For example, the vaccine comprises a whole cell vaccine, e.g., an allogeneic or autologous whole cell vaccine. In one aspect, the vaccine comprises an allogeneic PDA tumor cell engineered to secrete GM-CSF. For example, the vaccine comprises GM-CSF-secreting PDA vaccine (GVAX). GVAX is administered prior to, concurrently with, or subsequent to administration of the PD-1 inhibitor and/or the PD-L1 inhibitor.

Also provided are Listeria-based vaccines that express a gastrointestinal cancer-associated tumor antigen, e.g., a pancreatic cancer-associated tumor antigen. In one aspect, the vaccine comprises a Listeria monocytogenes (Lm)-based vaccine. For example, the Listeria monocytogenes (Lm)-based vaccine expresses an Annexin A2 (ANXA2) antigen. In a further example, the Listeria monocytogenes (Lm)-based vaccine expressing an Annexin A2 (ANXA2) antigen is administered prior to, concurrently with, or subsequent to administration of the PD-1 inhibitor and/or the PD-L1 inhibitor.

In another aspect, dendritic cell vaccines, i.e., dendritic cell therapies, are also useful in the methods described herein. The dendritic cell vaccines are administered prior to, concurrently with, or subsequent to administration of the PD-1 inhibitor and/or the PD-L1 inhibitor.

Exemplarly PD-1 inhibitors or PD-L1 inhibitors comprise an anti-PD-1 antibody, an anti-PD-L1 antibody, or combinations thereof. PD-1 inhibitors may be administered individually or in concert or sequential to the administration of PD-L1 inhibitors.

The vaccine and PD-1/PD-L1 inhibitor may further be administered prior to, in combination with, or sequential to other immune modulators and/or targeted therapies. Alternatively, the vaccine and PD-1/PD-L1 inhibitor is administered prior to, in combination with, or sequential to chemotherapy and/or chemoradiation.

PDA is classically considered a non-immunogenic tumor because very few effector T cells infiltrate these tumors. Preferably, the infiltration of CD8+ T lymphocytes, activated CD8+ T cells, and interferon gamma (IFNγ)-producing CD8+ T cells into PDA tumor microenvironment (TME) is increased following the administration of the compositions described herein.

In some cases, an immune modulating dose of cyclophosphamide is also administered to the subject prior to, concurrently with, or subsequent to GVAX treatment. In one aspect, cyclophosphamide is administered to the subject prior to, concurrently with, or subsequent to administration of the PD-1 inhibitor or the PD-L1 inhibitor. For example, the immune modulating dose of cyclophosphamide is 1 mg/kg, 10 mg/kg, 50 mg/kg, 100 mg/kg, 250 mg/kg, 500 mg/kg, 750 mg/kg, or 1 g/kg. Preferably, the cyclophosphamide is administered intraperitoneally or orally.

In some cases, the PD-1 inhibitor or the PD-L1 inhibitor is administered at a dosage of 0.01-10 mg/kg (e.g., 0.01, 0.05, 0.1, 0.5, 1, 5, or 10 mg/kg) bodyweight. For example, the PD-1 inhibitor or the PD-L1 inhibitor is administered in an amount of 0.01-30 mg (e.g., 0.01, 0.05, 0.1, 0.5, 1, 5, 10, 20, or 30 mg) per dose. In another example, the PD-1 inhibitor or the PD-L1 inhibitor is administered in the dose range of 0.1 mg/kg to 10 mg/kg of body weight.

In one aspect, GVAX vaccine cells are administered at a concentration of 10²/mL-10¹²/ml, e.g., 10³/mL, 10⁴/mL, 10⁵/mL, 10⁶/mL, 10⁷/mL, 10⁸/mL, 10⁹/mL, 10¹⁰/mL, or 10¹¹/mL. The total volume of GVAX administered is about 0.001 mL-about 10 mL, e.g., 0.01 mL, 0.1 mL, or 1 mL. For example, GVAX vaccine cells are administered at a concentration of 2×10⁷/mL in a total volume of 0.1 mL. Preferably, the GVAX vaccine is administered subcutaneously.

In some cases, the Listeria monocytogenes-based vaccine expressing an Annexin A2 (ANXA2) antigen is administered at a dosage of 1×10⁴ to 1×10¹² colony forming units (CFU), e.g., 10⁵, 10⁶, 10⁷, 10⁸, 10⁹, 10¹⁰, or 10¹¹ CFU. Preferably, the Lm-based vaccine is administered orally.

Regulatory T cells (Tregs) and cytotoxic T lymphocyte antigen-4 (CTLA-4) expression on T cells is inhibited, and the percentage of CD69+ CD8+ T cells among CD8+ lymphocytes infiltrating TME is increased compared to cyclophosphamide and GVAX alone. Subject survival time is increased compared to PD-1 monotherapy or GVAX monotherapy alone, and the PDA tumor is reduced or inhibited.

In some cases, the vaccine and/or the PD-1 inhibitor and/or the PD-L1 inhibitor is administered twice or more, e.g., 3 times, 4 times, 5 times, 6 times, 7 times, 8 times, 9 times, 10 times, 15 times, 20 times, 25 times, 30 times, 35 times, 40 times, 50 times, 60 times, 70 times, 80 times, 90 times or more. The cyclophosphamide is administered twice or more, e.g., 3 times, 4 times, 5 times, 6 times, 7 times, 8 times, 9 times, 10 times, 15 times, 20 times, 25 times, 30 times, 35 times, 40 times, 50 times, 60 times, 70 times, 80 times, 90 times or more. For example, the vaccine and the PD-1 inhibitor or the PD-L1 inhibitor are administered at least once per week, e.g., at least twice per week, at least three times per week, at least four times per week, at least five times per week, at least six times per week, at least seven times per week. Alternatively, the vaccine and the PD-1 inhibitor or the PD-L1 inhibitor are administered at least once per day, e.g., at least twice per day, at least every eight hours, at least every four hours, at least every two hours, or at least every hour. The compositions of the invention (e.g., GVAX and PD-1 inhibitor or the PD-L1 inhibitor) are administered for a duration of 1 day, 2 days, 3 days, 4 days, 5 days, 6 days, 7 days, 2 weeks, 3 weeks, 4 weeks, five weeks, six weeks, 2 months, 3 months, 4 months, 5 months, 6 months, 7 months, 8 months, 9 months, 10 months, 11 months, 12 months, 2 years, 3 years, 4 years, 5 years or more. For example, the composition of the invention (e.g., GVAX and PD-1 inhibitor or the PD-L1 inhibitor) are administered one dose every two weeks for 4 to 6 weeks or until the disease is treated.

Optionally, the the vaccine and the PD-1 inhibitor or the PD-L1 inhibitor are administered simultaneously. Alternatively, the the vaccine and the PD-1 inhibitor or the PD-L1 inhibitor are administered sequentially.

Also provided herein are compositions for the treatment or prevention of cancer comprising a Listeria monocytogenes (Lm)-based vaccine that expresses an ANXA2 antigen. In one aspect, the composition further comprises a PD-1 inhibitor or a PD-L1 inhibitor. For example, the PD-1 inhibitor or the PD-L1 inhibitor comprises an anti-PD-1 antibody or an anti-PD-L1 antibody.

Treatment with immunotherapeutic methods or compositions described herein may be a stand-alone treatment, or may be one component or phase of a combination therapy regime, in which one or more additional therapeutic agents are also used to treat the patient.

Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “control” or “reference” is meant a standard of comparison. As used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

As used herein, “detecting” and “detection” are understood that an assay performed for identification of a specific analyte in a sample, e.g., an antigen in a sample or the level of an antigen in a sample. The amount of analyte or activity detected in the sample can be none or below the level of detection of the assay or method.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “polynucleotide” or “nucleic acid” as used herein designates mRNA, RNA, cRNA, cDNA or DNA. As used herein, a “nucleic acid encoding a polypeptide” is understood as any possible nucleic acid that upon (transcription and) translation would result in a polypeptide of the desired sequence. The degeneracy of the nucleic acid code is well understood. Further, it is well known that various organisms have preferred codon usage, etc. Determination of a nucleic acid sequence to encode any polypeptide is well within the ability of those of skill in the art.

As used herein, “isolated” or “purified” when used in reference to a polypeptide means that a polypeptide or protein has been removed from its normal physiological environment (e.g., protein isolated from plasma or tissue, optionally bound to another protein) or is synthesized in a non-natural environment (e.g., artificially synthesized in an in vitro translation system or using chemical synthesis). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

As used herein, “kits” are understood to contain at least one non-standard laboratory reagent for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ), respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Also provided are variable domain antigen-binding sequences derived from human antibodies. Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

A “humanized antibody” is generally considered to be a human antibody that has one or more amino acid residues introduced into it from a source that is non-human. These non-human amino acid residues are often referred to as “import” residues, which are typically taken from an “import” variable domain Humanization is traditionally performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting import hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

An “intact” antibody is one that comprises an antigen-binding site as well as a C_{L} and at least heavy chain constant domains, C_{H} 1, C_{H} 2 and C_{H} 3. The constant domains may be native sequence constant domains (e.g., human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870; Zapata et al., Protein Eng. 8(10): 1057-1062 [1995]); single-chain antibody molecules; and multispecific antibodies formed from antibody fragments.

The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc_{ε}RI.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H} 1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, an antibody that “internalizes” is one that is taken up by (i.e., enters) the cell upon binding to an antigen on a mammalian cell (e.g., a cell surface polypeptide or receptor). The internalizing antibody will of course include antibody fragments, human or chimeric antibody, and antibody conjugates. For certain therapeutic applications, internalization in vivo is contemplated. The number of antibody molecules internalized will be sufficient or adequate to kill a cell or inhibit its growth, especially an infected cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the infected cell.

As used herein, an antibody is said to be “immunospecific,” “specific for” or to “specifically bind” an antigen if it reacts at a detectable level with the antigen, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and in certain embodiments, HuM2e antibody specifically binds to M2e if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of antibodies can be readily determined using conventional techniques, for example, those described by Scatchard et al. (Ann. N.Y. Acad. Sci. USA 51:660 (1949)).

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody.

The term “antagonist” antibody is used in the broadest sense, and includes an antibody that partially or fully blocks, inhibits, or neutralizes a biological activity of an epitope, polypeptide, or cell that it specifically binds. Methods for identifying antagonist antibodies may comprise contacting a polypeptide or cell specifically bound by a candidate antagonist antibody with the candidate antagonist antibody and measuring a detectable change in one or more biological activities normally associated with the polypeptide or cell.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al., PNAS (USA) 95:652-656 (1998).

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42. degree. C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Obtaining” is understood herein as manufacturing, purchasing, or otherwise coming into possession of.

As used herein, “operably linked” is understood as joined, preferably by a covalent linkage, e.g., joining an amino-terminus of one peptide, e.g., expressing an enzyme, to a carboxy terminus of another peptide, e.g., expressing a signal sequence to target the protein to a specific cellular compartment; joining a promoter sequence with a protein coding sequence, in a manner that the two or more components that are operably linked either retain their original activity, or gain an activity upon joining such that the activity of the operably linked portions can be assayed and have detectable activity, e.g., enzymatic activity, protein expression activity.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intracardiac, intraperotineal, intrathecal, intracranial, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments). Optionally the peptide further includes one or more modifications such as modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins, Structure and Molecular Properties, 2nd ed., T. E. Creighton, W.H. Freeman and Company, New York (1993); Posttranslational Covalent Modification of Proteins, B. C. Johnson, ed., Academic Press, New York, pgs. 1-12 (1983); Seifter et al., Meth. Enzymol 182:626-646 (1990); Rattan et al., Ann. N.Y. Acad. Sci. 663:48-62 (1992)).

The term “reduce” or “increase” is meant to alter negatively or positively, respectively, by at least 5%. An alteration may be by 5%, 10%, 25%, 30%, 50%, 75%, or even by 100%.

A “sample” as used herein refers to a biological material that is isolated from its environment (e.g., blood or tissue from an animal, cells, or conditioned media from tissue culture) and is suspected of containing, or known to contain an analyte, such as a protein. A sample can also be a partially purified fraction of a tissue or bodily fluid. A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “subject” as used herein refers to an organism. In certain embodiments, the organism is an animal. In certain embodiments, the subject is a living organism. In certain embodiments, the subject is a cadaver organism. In certain preferred embodiments, the subject is a mammal, including, but not limited to, a human or non-human mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A “subject sample” can be a sample obtained from any subject, typically a blood or serum sample, however the method contemplates the use of any body fluid or tissue from a subject. The sample may be obtained, for example, for diagnosis of a specific individual for the presence or absence of a particular disease or condition.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer is within the ability of those in the art. Subjects suffering from, and suspected of suffering from, a specific disease, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Unless specifically stated or obvious from context, as used herein, the term “or” is understood to be inclusive. Unless specifically stated or obvious from context, as used herein, the terms “a”, “an”, and “the” are understood to be singular or plural.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A and FIG. 1B are a series of photomicrographs demonstrating that pancreatic cancer Cy/GVAX therapy upregulates pancreatic tumor expression of PD-L1 in human and murine PDA. FIG. 1A shows representative hematoxylin and eosin (H&E) staining and IHC with αPD-L1 antibodies on resected PDA from unvaccinated patients and patients who received the Cy/GVAX vaccine therapy 2 weeks before surgical resection. FIG. 1B shows immunofluorescence staining with αPD-L1 antibodies and FITC conjugated secondary antibody in liver tumors after Panc02 hemispleen injection comparing mice treated with Cy/GVAX on day 4 and 7 after tumor inoculation (w/GVAX) against mice not receiving GVAX therapy (w/o GVAX; upper panel). Livers were harvested 2 weeks after tumor cell inoculation. DAPI (4′,6-diamidino-2-phenylindole) staining of nuclei is shown in lower panel.

FIG. 2A-FIG. 2E is a schematic, a series of line graphs and a series of bar charts demonstrating that combination therapy with Cy/GVAX and PD-1 or PL-L1 blockade improves clinical outcomes in a PDA mouse model. FIG. 2A is a schematic of tumor implantation by the hemispleen procedure and treatment with Cy, GVAX and αPD-1/αPD-L1 blockade as indicated. C57B1/6 mice were challenged on day 0 with 2×10⁶ Panc02 tumor cells followed by administration of 100 mg/kg of Cy on day 3 and irradiated whole-cell vaccine on days 4, 7, 14, and 21. Anti-PD-1, anti-PD-L1, or IgG (5 mg/kg IP) were administered IP twice weekly until death starting on day 3. FIG. 2B is a series of Kaplan-Meier survival curves of mice that were implanted with PDA cells and were treated with different combinations of Cy, GVAX and the αPD-1 antibody. IgG, hamster IgG. Cy, cyclophosphamide. FIG. 2C and FIG. 2D are bar charts showing the percentages of mice that remained disease free at day 90 following tumor implantation and therapy with Cy, GVAX, and/or αPD-1 (FIG. 2C) or Cy, GVAX, and aPL-L1 (FIG. 2D). All the p values were yielded by comparing GVAX and/or αPD-1/αPD-L1 treatment groups with IgG treated group. FIG. 2E is a series of Kaplan-Meier survival curves of mice that were implanted with Panc02 cells via hemispleen technique and treated with different combinations of Cy, GVAX, and αPD-L1 antibody. Data are represented as results obtained from experiments with 8 to 10 mice per group that were repeated at least twice. N.S. not significant.

FIG. 3A-FIG. 3E is a schematic, a series of dot plots, and a series of bar charts showing that Cy/GVAX combined with PD-1 blockade increases CD8⁺ T cells in PDAs. FIG. 3A is a schematic of immune analysis following tumor implantation by the hemislpeen procedure and treatment with Cy (100 mg/kg) on day 3, GVAX on day 4, 7, and IgG/αPD-1/αPD-L1 (5 mg/kg IP) on days 3, 6, 10. Each experimental group consisted of 5 mice, pooled and analyzed individually in triplicates. FIG. 3B and FIG. 3C show the percentage of CD8⁺ (FIG. 3B) and CD4⁺ (FIG. 3C) tumor-infiltrating lymphocytes among total lymphocytes in murine livers, while FIG. 3D and FIG. 3E show total numbers of CD8⁺ (FIG. 3D) and CD4⁺ (FIG. 3E) tumor-infiltrating lymphocytes after Panc02 hemispleen and indicated therapy. Data represent mean +/− SEM from 1 representative experiment that was repeated at least twice. *P=0.04. TIL: tumor-infiltrating lymphocytes.

FIG. 4A-FIG. 4D is a series of dot plots and a series of bar charts demonstrating that combinatorial treatment increases the percentage of interferon (IFN)γ-secreting CD8+ T cells and tumor-specific CD8+ T cells in the tumor microenvironment. CD8+ T cells were isolated and purified from spleen and livers on day 13 after hemispleen implantation of Panc02 tumor cells. Tumor-bearing mice were treated with Cy, GVAX, or αPD-1/αPD-L1 therapy as indicated. The percentage of IFNγ+-producing CD8+ T cells among all CD8+ T cells in (FIG. 4A) splenocytes and (FIG. 4B) tumor-infiltrating lymphocytes is shown. ELISA assays were performed using autologous irradiated Panc02 tumor cells as antigenic targets for CD8+ T cells isolated from (FIG. 4C) spleen and (FIG. 4D) tumor-infiltrating lymphocytes. Each experimental group consisted of 5 mice, pooled and analyzed individually in triplicates. Data represent mean±SEM from 1 representative experiment that was repeated at least twice. *P<0.05, **P<0.01, ***P<0.001.

FIG. 5A-FIG. 5I are a series of plots and bar graphs showing that Cy/GVAX therapy with αPD-1 blockade overcomes immunosuppressive pathways. Following hemispleen implantation of Panc02 cells, tumor-bearing mice were treated with Cy (100 mg/kg) on day 3, GVAX on day 4, 7, and IgG/αPD-1/αPD-L1 (5 mg/kg IP) on days 3, 6, 10. Mice were killed on day 13. FIG. 5A is a series of fluorescence-activated cell sorting (FACS) density plots of CD4+ CD25+ Foxp3+ Tregs in TILs. FIG. 5B is a histogram showing the percentage of CD4+ CD25+ Foxp3+ Tregs in CD4+ TILs. FIG. 5C is a histogram showing the percentage of CD4+ CD25+ Foxp3+ Tregs in all TILs. FIG. 5D is a histogram showing the total number of CD4+ CD25+ Foxp3+ Tregs infiltrating the tumors. FIG. 5E is a series of representative FACS analysis of CTLA-4+ CD8+ T cells in TILs. FIG. 5F is a histogram showing the percentage of CTLA-4+ CD8+ T cells within CD8+ T cells. FIG. 5G is a histogram showing the percentage of CTLA-4+ CD4+ T cells within CD4+ T cells. FIG. 5H is a histogram showing the total number of CTLA-4+ CD8+ TILs. FIG. 5I is a histogram showing the total number of CTLA-4+ CD4+ TILs. Each experimental group consisted of 5 mice, pooled and analyzed individually in triplicates. Data represent mean±SEM from 1 representative experiment that was repeated at least twice. *P<0.05, **P<0.01, ***P<0.001.

FIG. 6 is a photomicrograph showing representative H&E staining of tumors formed by Panc02 pancreatic tumor cells in murine liver.

FIG. 7A-FIG. 7B is a series of bar charts. After hemispleen implantation of Panc02 cells, tumor-bearing mice were treated with Cy (100 mg/kg) on day 3, GVAX on day 4, 7 and IgG/αPD-1/αPD-L1 (100 ug IP) on day 3, 6, 10. Mice were sacrificed on day 13 and the percentage of (FIG. 7A) CD8⁺ and (FIG. 7B) CD4⁺ splenocytes within all splenic lymphocytes after Panc02 hemispleen implantation was calculated. Each experimental group consisted of five mice, pooled and analyzed individually in triplicates. Data represent mean±SEM from one representative experiment that was repeated at least twice. *p<0.05, **p<0.01, ***p<0.001.

FIG. 8A-FIG. 8B is a series of bar charts. After hemispleen implantation of Panc02 cells, tumor-bearing mice were treated with Cy (100 mg/kg) on day 3, GVAX on day 4, 7 and IgG/αPD-1/αPD-L1 (100 ug IP) on day 3, 6, 10. Mice were sacrificed on day 13 and total numbers of IFNγ⁺ producing CD8⁺ T cells in the hemispleen (FIG. 8A) and liver (FIG. 8B) were evaluated. Each experimental group consisted of five mice, pooled and analyzed individually in triplicates. Data represent mean±SEM from one representative experiment that was repeated at least twice. ***p<0.001, ****p<0.0001.

FIG. 9A-FIG. 9D is a series of dot plots an a bar chart. CD8⁺ T cells were isolated and purified from spleen and livers on day 13 after hemispleen implantation of Panc02 tumor cells. Tumor-bearing mice were treated Cy, GVAX or αPD-1/αPD-L1 therapy as indicated. Activated tumor-infiltrating lymphocytes in PDA liver metastases designated as (FIG. 9A) percentages of CD69⁺ CD8⁺ T cells among total lymphocytes in murine livers, (FIG. 9B) percentages of CD69⁺ CD8⁺ T cells among CD8⁺ TIL, (FIG. 9C) absolute numbers of CD69⁺ CD8⁺ T cells in murine livers, and (FIG. 9D) CD69⁺ CD4⁺ T cells among total lymphocytes in murine livers are shown.

FIG. 10A-FIG. 10C is a series of bar charts After hemispleen implantation of Panc02 cells, tumor-bearing mice were treated with Cy (100 mg/kg) on day 3, GVAX on day 4, 7 and IgG/αPD-1/αPD-L1 (100 ug IP) on day 3, 6, 10. Mice were sacrificed on day 13 and splenocytes were evaluated for the (FIG. 10A) percentage of FoxP3⁺ expressing CD25⁺ CD4⁺ regulatory T cells within all CD4⁺ T cells, (FIG. 10B) the percentage of CTLA-4⁺ CD8⁺ T cells in CD8 splenocytes and (FIG. 10C) CTLA-4⁺ CD4⁺ T cells in CD4⁺ splenocytes. Each experimental group consisted of five mice, pooled and analyzed individually in triplicates. Data represent mean±SEM from one representative experiment that was repeated at least twice.

### DETAILED DESCRIPTION

The invention is based, at least in part, on the surprising discovery that PD-1/PD-L1 blockade together with vaccine therapy facilitates effector T cell infiltration into pancreatic tumors. Specifically, combinatorial therapy with GM-CSF-secreting PDA vaccine (GVAX) and PD-1 antibody and/or PD-L1 antibody blockade improved murine survival compared to PD-1 antibody monotherapy or GVAX therapy alone.

Pancreatic ductal adenocarcinoma (PDA) has a poor prognosis due to late detection and resistance to conventional therapies. Published results show that the PDA tumor microenvironment (TME) is predominantly infiltrated with immune suppressive cells and signals that if altered allow effective immunotherapy. However, single-agent checkpoint inhibitors including agents that alter immune suppressive signals in other human cancers including cytotoxic T lymphocyte antigen-4 (CTLA-4), programmed death 1 (PD-1) and it's ligand PD-L1, have failed to demonstrate objective responses when given as single agents to PDA patients Inhibition of the CTLA-4 pathway when given together with a T cell inducing vaccine gives objective responses in metastatic PDA patients. Described in detail below is the evaluation of blockade of the PD-1/PD-L1 pathway.

As described below, PD-L1 is weakly expressed at a low frequency in untreated human and murine PDAs; however, treatment with a GM-CSF-secreting PDA vaccine (GVAX) significantly upregulates PD-L1 membranous expression in pancreatic tumors after treatment of tumor bearing mice. In addition, combination therapy with vaccine and PD-1 antibody blockade improved murine survival compared with PD-1 antibody monotherapy or GVAX therapy alone. Furthermore, PD-1 blockade increased effector CD8+ T lymphocytes and tumor-specific interferon-γ production of CD8+ T cells in the tumor microenvironment. Immunosuppressive pathways, including regulatory T cells and CTLA-4 expression on T cells were overcome by the addition of vaccine and low-dose cyclophosphamide to PD-1 blockade. Collectively, these findings support combining PD-1 or PD-L1 antibody therapy with a T cell inducing agent for PDA treatment.

Pancreatic Ductal Adenocarcinoma

Pancreatic ductal adenocarcinoma (PDA) is the fourth leading cause of cancer related deaths in the United States (Siegel et al., 2014 Cancer Statistics, 64(1):9-29). Over 80% of those diagnosed with PDA are ineligible for curative resection and five-year survival is less than 5% (Moon et al., 2006 Pancreas, 32(1):37-43; Ma et al., 2013 Journal of the National Cancer Institute, 105(22):1694-700).

The symptoms at diagnosis vary according to the location of the cancer in the pancreas, which anatomists divide (from left to right on most diagrams) into the thick head, the neck, and the tapering body, ending in the tail. Regardless of a tumor's location, the most common symptom is unexplained weight loss, which may be considerable. A large minority (between 35% and 47%) of people diagnosed with the disease will have had nausea, vomiting or a feeling of weakness. Tumors in the head of the pancreas typically also cause jaundice, pain, loss of appetite, dark urine, and light-colored stools. Tumors in the body and tail typically also cause pain. People sometimes have recent onset of atypical type 2 diabetes that is difficult to control, a history of recent but unexplained blood vessel inflammation caused by blood clots (thrombophlebitis) known as Trousseau sign, or a previous attack of pancreatitis. A physician may suspect pancreatic cancer when the onset of diabetes in someone over 50-years-old is accompanied by typical symptoms such as unexplained weight loss, persistent abdominal or back pain, indigestion, vomiting, or fatty feces. Jaundice accompanied by a painlessly swollen gallbladder (known as Courvoisier's sign) may also raise suspicion, and can help differentiate pancreatic cancer from gallstones.

Medical imaging techniques, such as computed tomography (CT scan) and endoscopic ultrasound (EUS) are used both to confirm the diagnosis and to help decide whether the tumor can be surgically removed. Magnetic resonance imaging and positron emission tomography may also be used, and magnetic resonance cholangiopancreatography may be useful in some cases. Abdominal ultrasound is less sensitive and will miss small tumors, but can identify cancers that have spread to the liver and build-up of fluid in the peritoneal cavity (ascites). A biopsy by fine needle aspiration, often guided by endoscopic ultrasound, may be used where there is uncertainty over the diagnosis. Liver function tests can show a combination of results indicative of bile duct obstruction (raised conjugated bilirubin, γ-glutamyl transpeptidase and alkaline phosphatase levels).

The most common form of pancreatic cancer (adenocarcinoma) is typically characterized by moderately to poorly differentiated glandular structures on microscopic examination. There is typically considerable desmoplasia or formation of a dense fibrous stroma or structural tissue consisting of a range of cell types (including myofibroblasts, macrophages, lymphocytes and mast cells) and deposited material (such as type I collagen and hyaluronic acid). This creates a tumor microenvironment that is short of blood vessels (hypovascular) and oxygen (tumor hypoxia).

Current treatment modalities, including surgical resection, chemotherapy, and radiation have failed to significantly improve PDA survival in the last 30 years. Thus, prior to the invention described herein, new treatment modalities were desperately needed (Ma et al., 2013 Journal of the National Cancer Institute, 105(22):1694-700).

Immunotherapy has shown promise against solid tumors such as melanoma, renal cell carcinoma (RCC), non-small lung cancer (NSLC) and prostate cancer (Antonarakis E S, Drake C G, 2010 Current opinion in urology, 20(3):241-6; Sharma et al., 2012 Nat Rev Cancer, 11(11):805-12; Rosenberg et al., 1988 The New England journal of medicine, 988;319(25):1676-80). PDA is classically considered a non-immunogenic tumor because very few effector T cells infiltrate these tumors (Zheng et al., 2013 Gastroenterology, 144(6):1230-40; Vonderheide R H, Bayne L J, 2013 Current opinion in immunology, 25(2):200-5). Although a survival benefit was seen in surgically resected PDA patients with higher levels of CD4 and CD8 tumor infiltrating lymphocytes (TIL) within the tumor microenvironment (TME) (Fukunaga et al., 2004 Pancreas, 28(1):e26-31), the majority of PDAs contain a strong immunosuppressive network which limits the immune system's ability to actively eradicate the disease (Ene-Obong et al., 2013 Gastroenterology, 145(5):1121-32). The development of PDA is associated with alterations in its TME from pro-inflammatory to tolerogenic, characterized by infiltration of immunosuppressive cells such as regulatory T cells (Tregs) and myeloid derived suppressive cells (MDSCs) along with pro-cancerous inflammatory signals (Zheng et al., 2013 Gastroenterology, 144(6):1230-40; Bayne et al., 2012 Cancer cell, 21(6):822-35; Clark et al., 2007 Cancer research, 67(19):9518-27).

Program death receptor-1 (PD-1) and one of its major ligands, program death ligand 1 (PD-L1), constitute a major tolerance mechanism (Flies D B and Chen L, 2007 Journal of Immunotherapy, 30(3):251-60). PD-L1 (or B7-H1) is expressed by tumor cells, antigen presenting cells, B cells, and parenchymal cells and binds to PD-1 which is mainly expressed on activated T cells (Keir Meet al., 2008 Annual review of immunology, 26:677-704; Francisco et al., 2010 Immunological reviews, 236:219-42). The binding of PD-L1 to PD-1 results in T cell anergy or death, thereby blunting anti-tumor immune responses and promoting tumor growth (Keir Meet al., 2008 Annual review of immunology, 26:677-704; Dong et al., 2002 Nature medicine, 8(8):793-800). The expression of PD-1/PD-L1 has been characterized in PDAs (Loos et al., 2008 Cancer letters, 268(1):98-109; Nomi et al., 2007 Clinical Cancer Research: an official journal of the American Association for Cancer Research, 13(7):2151-7; Batra et al., 1991 Cell growth & differentiation: the molecular biology journal of the American Association for Cancer Research, 2(8):385-90; Basso et al., 2013 PloS one, 8(1):e54824; Wang et al., 2010 World Journal of Surgery, 34(5):1059-65; Geng et al., 2008 Journal of Cancer Research and Clinical Oncology, 134(9):1021-7). Despite some reports correlating PD-L1 expression with a poorer prognosis, prior to the invention described herein, the overall knowledge on the role of this pathway in PDA is still limited (Nomi et al., 2007 Clinical Cancer Research: an official journal of the American Association for Cancer Research, 13(7):2151-7). When αPD-L1 antibodies were tested in clinical trials for a limited number of metastatic PDA patients, no objective responses were seen (Brahmer et al., 2012 The New England Journal of Medicine, 366(26):2455-65). However, clinical trials of αPD-1 and αPD-L1 antibodies demonstrated durable tumor regression and prolonged stabilization of disease in NSCLC, melanoma and RCC (Brahmer et al., 2012 The New England Journal of Medicine, 366(26):2455-65; Hamid O, Robert C, Daud A, Hodi F S, Hwu W J, Kefford R, et al. 2013 The New England Journal of Medicine, 369(2):134-44; Brahmer et al., 2010 Journal of Clinical Oncology: official journal of the American Society of Clinical Oncology, 28(19):3167-75; Topalian et al., 2012 The New England Journal of Medicine, 366(26):2443-54).

A possible explanation for the therapeutic failure of PD-1 or PD-L1 blockade therapy in PDA is the lack of a natural infiltration of effector immune cells in the majority of PDAs. A potential strategy to activate effector T cell trafficking into the TME is vaccine-based immunotherapy. A human whole cell granulocyte macrophage colony-stimulating factor (GM-CSF) secreting pancreatic cancer vaccine (GVAX) composed of allogeneic PDA tumor cell lines engineered to secrete GM-CSF has been developed (Jaffee et al., 2001 Journal of Clinical Oncology: official journal of the American Society of Clinical Oncology, 19(1):145-56, incorporated herein by reference). Phase I and II clinical trials of this vaccine demonstrated its safety and its ability to enhance tumor antigen (mesothelin)-specific interferon-γ (IFN-γ) producing T cells in peripheral lymphocytes, which correlated with prolonged survival (Jaffee et al., 2001 Journal of Clinical Oncology: official journal of the American Society of Clinical Oncology, 19(1):145-56; Laheru et al., 2008 Clinical Cancer Research: an official journal of the American Association for Cancer Research, 14(5):1455-63; Lutz et al., 2011 Annals of Surgery, 253(2):328-35; Thomas et al., 2004 The Journal of Experimental Medicine, 200(3):297-306; Le et al., 2013 Journal of Immunotherapy, 36(7):382-9; Schueneman et al., 2013 Annals of Surgical Oncology, 20 Suppl 3:725-30, each of which is incorporated herein by reference). A recent clinical trial of GVAX as a neoadjuvant therapy for resectable PDAs identified vaccine-induced tertiary lymphoid aggregates in PDAs surgically resected from the majority of patients who received the vaccine therapy 2 weeks before the surgery as well as the infiltration of PD-L1⁺ cells within these lymphoid aggregates (Lutz et al. 2014 Cancer Immunol Res, 2: 616-631, incorporated herein by reference). GVAX also indues infiltration of IFNγ-producing T cells in PDAs.

Similar to αPD-1 and αPD-L1, ipilimumab therapy, which is a checkpoint blockade antibody against cytotoxic T lymphocyte antigen-4 (CTLA-4) approved by the United States Food and Drug Administration (FDA) for the treatment of unresectable melanoma (Hodi et al., 2010 The New England Journal of Medicine, 363(8):711-23; Robert et al., 2011 The New England Journal of Medicine, 364(26):2517-26), failed to demonstrate durable and effective anti tumor activity in metastatic PDA patients as a single agent. However, the combination of ipilimumab with PDA GVAX demonstrated objective clinical responses that were associated with prolonged survival when compared to single agent ipilimumab (Le et al., 2013 Journal of Immunotherapy, 36(7):382-9) Immune related adverse effects are common and severe with ipilimumab therapy in contrast to those seen with PD-1/PD-L1 blockade, which are less frequent and more manageable (Brahmer et al., 2012 The New England Journal of Medicine, 366(26):2455-65; Topalian et al., 2012 The New England Journal of Medicine, 366(26):2443-54; Gangadhar T C and Vonderheide R H, 2014 Nature Reviews Clinical Oncology, 11(2):91-9). Therefore, the effect of the combination of GVAX with PD-1/PD-L1 blockade therapies in a PDA mouse model was examined as described in detail below.

Mechanistically, PD-L1/PD-1 and CTLA-4 function differently in T cell regulation (Topalian et al., 2012 Current Opinion in Immunology, 24(2):207-12). Although PD-L1 expression can be induced by oncogenic signals, it is mainly activated by adaptive immune responses (Pardoll D M, 2012 Nat Rev Cancer, 12(4):252-64). Spranger showed that the up-regulation of PD-L1 in the TME was dependent on CD8⁺ T cells and IFN-γ (Spranger et al., 2013 Science Translational Medicine, 5(200):200ra116). Taube demonstrated that 98% of PD-L1-expressing melanomas were associated with tumor-infiltrating lymphocytes (TILs) as opposed to PD-L1 negative tumors where only 28% of these were associated with TIL presence (Taube et al., 2012 Science Translational Medicine, 4(127):127ra37). Moreover, PD-L1 expression on the tumor was preferentially seen at the tumor-TIL interface correlating with the presence of IFNγ (Taube et al., 2012 Science Translational Medicine, 4(127):127ra37). These findings suggest that upregulation of PD-L1 expression within the TME in response to an endogenous anti-tumor immune response subsequently generates an immune checkpoint signal, a process termed adaptive resistance (Topalian et al., 2012 The New England Journal of Medicine, 366(26):2443-54; Topalian et al., 2012 Current Opinion in Immunology, 24(2):207-12; Pardoll D M, 2012 Nat Rev Cancer, 12(4):252-6439; Taube et al., 2012 Science Translational Medicine, 4(127):127ra37). Therefore, whether similar mechanisms underlie tolerance to vaccine-based cancer immunotherapy and whether PD-1 or PD-L1 blockade therapy can overcome this tolerance mechanism was examined as described in detail below.

As described in detail below, baseline PD-L1 levels in mouse and human PDAs were determined. It was also determined whether PD-L1 mediated adaptive resistance occurs in PDAs following vaccine-based immunotherapy in mouse PDAs, and whether αPD-1 or αPD-L1 antibody treatment in combination with GVAX induces a greater anti-tumor immune response than either immunotherapy alone.

Recent studies support the concept of adaptive resistance mediated by PD-L1/PD-1 signaling. PD-L1 expression can be induced by inflammatory cells and cytokines, particularly IFN-γ (Dong et al., 2002 Nature Medicine, 8(8):793-800; Loos et al., 2008 Cancer letters, 268(1):98-109, Spranger et al., 2013 Science Translational Medicine, 5(200):200ra116; Taube et al., 2012 Science Translational Medicine, 4(127):127ra37; Curiel et al., 2003 Nature Medicine, 9(5):562-7). PDA, like most gastrointestinal cancers, is considered non-immunogenic most likely secondary to their immune inhibitory TME. αPD-1/αPD-L1 monotherapy's lack of an antitumor immune response against PDA suggests that the PD-1/PDL1 pathway may not play a natural role in the immune regulation of these tumors. Alternatively, PD-1/PD-L1 signaling may be involved in the early stage of PDA development and subsequently become dispensable after other tolerance mechanisms have been activated. The data presented herein in both murine and human PDAs confirm that there is low expression of the PD-L1 signal in PDA tumors.

As described in detail below, PD-L1 expression can be augmented in murine PDAs when IFNγ-producing CD8⁺ T cells infiltrate the TME. Similar findings were identified in a recently completed neo-adjuvant study in which patients received GVAX 2 weeks prior to surgical resection of their PDA in which the majority of patients developed PD-L1 expressing lymphoid aggregates. Unlike RCC, melanoma, and some NSLC, few IFN-γ-producing CD8⁺ T cells naturally reside in PDAs, but can be recruited by GVAX, which induces antigen-specific T cells that recognize mesothelin, a tumor antigen expressed by the majority of PDAs, and that has correlated with a survival advantage in patients in several trials (Jaffee et al., 2001 Journal of Clinical Oncology: official journal of the American Society of Clinical Oncology. 2001; 19(1):145-56; Laheru et al., 2008 Clinical Cancer Research: an official journal of the American Association for Cancer Research, 14(5):1455-63; Lutz et al., 2011 Annals of Surgery, 253(2):328-35; Thomas et al., 2004 The Journal of Experimental Medicine, 200(3):297-306; Le et al., 2013 Journal of Immunotherapy, 36(7):382-9). These data support that PD-L1 expression within the TME is in response to an endogenous anti tumor immune response. Infiltrating T cells are required to induce this particular immune checkpoint signal, supporting the proposition that the process of adaptive resistance can occur in PDA. Although few IFN-γ-expressing T cells were observed in treatment of naïve murine and human tumors, a PDA tumor-TIL interface is difficult to define because pancreatic neoplastic cells, in contrast to melanoma, are scattered within a dense stroma which is diffusely infiltrated with inflammatory cells, the majority of which are suppressive monocytes and Tregs.

The results presented herein show that the GVAX induced IFN-γ-expressing T cells that infiltrate the PDA TME selectively utilize the PD-1/PD-L1 pathway to mediate vaccine induced immune suppression of antigen-specific T cells. Unlike PD-L1, CTLA-4 and Tregs, two other regulatory mechanisms known to regulate antigen-specific T cells, were downregulated. As described in detail below, the Cy administered to deplete Tregs does effectively reduce their numbers and therefore, their influence of antigen-specific T cell suppression. In view of recent findings, it is also likely that reduction of CTLA-4 signaling on these T cells results from the reduction in Tregs (Simpson et al., 2013 The Journal of Experimental Medicine, 210(9):1695-710). CTLA-4 is likely still important especially in light of evidence demonstrating objective responses in metastatic PDA patients treated with GVAX and ipilimumab.

Other studies reported that there is minimal constitutive PD-L1 expression on Panc02 murine tumor cells, but increased PD-L1 expression occurs when these tumor cells are stimulated with IFN-γ (Nomi et al., 2007 Clinical Cancer Research: an official journal of the American Association for Cancer Research, 13(7):2151-7; Okudaira et al., 2009 International Journal of Oncology, 35(4):741-9). Some also reported delayed tumor growth in mouse models of PDA with αPD-L1 monotherapy; however, αPD-L1 single agent therapy did not eradicate tumors that were either implanted subcutaneously or orthotopically in the pancreas (Nomi et al., 2007 Clinical Cancer Research: an official journal of the American Association for Cancer Research, 13(7):2151-7; Okudaira et al., 2009 International Journal of Oncology, 35(4):741-9). αPD-L1 therapy did not demonstrate any objective responses as a single agent in metastatic PDAs in early phase clinical trials further confirming that PDA has a different inflammatory milieu when compared with NSCLC, melanoma, and RCC which do respond to single agent therapy (Brahmer et al., 2012 The New England Journal of Medicine, 366(26):2455-65).

Consistent with these findings, described herein is the effect of αPD-1 monotherapy in prolonging survival and curing a small percentage of metastasis bearing mice. However, as described in detail below, overall survival and cure rates were significantly improved when Cy/GVAX was combined with αPD-1 or αPD-L1 blockade. Thus, the results presented herein demonstrate that a T cell augmenting agent that facilitates trafficking of IFN-γ-expressing T cells into the PDA TME should be given with both αPD-1/αPD-L1 blockade to demonstrate significant clinical activity in patients with metastatic PDA. In accordance with the results presentd herein, previous studies have documented the additive benefit of combining vaccine therapy with PD-1 or PD-L1 blockade in other tumor models (Duraiswamy et al., 2013 Cancer Research, 73(23):6900-12; Duraiswamy et al., 2013 Cancer Research, 73(12):3591-603; Li et al., 2009 Clinical Cancer Research: an official journal of the American Association for Cancer Research, 15(5):1623-34; Mkrtichyan et al., 2011 European Journal of Immunology, 41(10):2977-86). However, in both a CT26 colon tumor and ID8 ovarian tumor subcutaneous model, αPD-1/αPDL1 or αCTLA-4 monotherapy led to significantly more effector T cell immune responses and antitumor activity than αPD-1/αPD-L1 did in the liver metastasis model of PDA (Duraiswamy et al., 2013 Cancer Research, 73(12):3591-603). The differences in treatment response are likely attributed to the difference in the tumor types. Both αPD-1/αPD-L1 monotherapies were associated with objective responses in colorectal and ovarian cancer patients, but were not effective in metastatic PDA patients in clinical trials. Additionally, the liver metastasis model is a physiologically relevant model of PDAs, as the liver is the most common site of metastasis for PDAs and the majority of PDA patients have metastatic disease at the time of diagnosis. As the liver is also the most common site of colorectal cancer metastases, αPD-1/αPD-L1 therapies in the liver metastasis model of CT26 cells is evaluated as described herein (Jain et al., 2003 Annals of Surgical Oncology, 10(7):810-20.

As presented herein, αPD-1/αPD-L1 monotherapy enhanced the infiltration of Tregs in the tumors formed by Panc02 cells. However, others have shown that αPD-1/αPD-L1 monotherapy reduces Tregs in the tumors formed by CT26 or ID8 cells (Duraiswamy et al., 2013 Cancer Research, 73(12):3591-603). Loos has also shown that PD-L1 positive PDAs demonstrate increased prevalence of regulatory T cells compared to PD-L1 negative tumors (Loos et al., 2008 Cancer letters, 268(1):98-109). The exact reasons for this difference is unclear, but might provide one explanation for why PDA patients do not respond to αPD-1/αPD-L1 monotherapy compared with patients who have other tumor types. Regardless, the results presented herein demonstrate that the PD-1/PD-L1 pathway is important in regulating antigen-specific T cell infiltration and function within the PDA TME once they are induced, and that combining αPD-1 or αPD-L1 blockade with Cy/GVAX that induce antigen specific T cells results in durable tumor control in mice with PDA.

The immune modulating dose of Cy is a critical component of this treatment regimen. As described herein, αPD-1 monotherapy significantly upregulated the prevalence of Tregs in the TME. A similar upregulation of Tregs in the TME when GVAX is given without Cy was also observed. Moreover, the addition of Cy to GVAX therapy has been previously shown to induce the recruitment of high avidity CD8⁺ T cells, which was attributed to Treg depletion (Ercolini et al., 2005 The Journal of Experimental Medicine, 201(10):1591-602). Previous reports have studied the effectiveness of GVAX in the treatment of murine colorectal cancer hepatic metastases and demonstrated that Cy in conjunction with CT26 GVAX resulted in transient depletion of Tregs as well as expansion of tumor antigen specific T cells (Jain et al., 2003 Annals of Surgical Oncology, 10(7):810-20; Radojcic et al., 2010 Cancer Immunology, Immunotherapy: CII, 59(1):137-48). The results presented herein also show that the addition of immune modulating dose of Cy together with GVAX depletes Tregs allowing for the enhanced effect of effector/activated T cells in the setting of a vaccine. Moreover, Cy/GVAX had a more significant effect in suppressing CTLA-4⁺ T cells than αPD-1 blockade. The role of Cy with or without GVAX and the role of GVAX with or without Cy was not within the scope of this study. However, it is likely that the inhibitory effects of Cy/GVAX on Tregs and CTLA-4 expression are derived from Cy. Nonetheless, an enhancement in antitumor activity was not observed with PD-1/PD-L1 blockade and a single intravenous dose of Cy alone, or as given together with GVAX. Most of the preclinical and clinical data support the combination of Cy and vaccines (Walter et al., 2012 Nature Medicine, 18(8):1254-61).

In conclusion, the results presented herein provide evidence of therapy-induced adaptive resistance with induction of PD-L1 expression in a murine PDA. The evidence supports the addition of αPD1 or αPD-L1 blockade to GVAX-based immunotherapy to achieve durable tumor responses. Adding Cy/GVAX therapy to αPD-1 or αPD-L1 therapy also overcomes additional immune checkpoint mechanisms.

Pharmaceutical Compositions and Administration

The present invention comprises pharmaceutical preparations comprising a vaccine (e.g., a granulocyte macrophage colony-stimulating factor (GM-CSF)-secreting pancreatic ductal adenocarcinoma (PDA) vaccine (GVAX) and a programmed death 1 (PD-1) inhibitor or a PD-1 ligand (PD-L1) inhibitor together with a pharmaceutically acceptable carrier. Such compositions are useful for the treatment or prevention of cancer, e.g., PDA. Polypeptides of the invention may be administered as part of a pharmaceutical composition. The compositions should be sterile and contain a therapeutically effective amount of the polypeptides in a unit of weight or volume suitable for administration to a subject.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups also can be derived from inorganic bases such as, for example, sodium, potassium, ammonium, calcium or ferric hydroxides, and such organic bases as isopropylamine, trimethyl amine, 2-ethylamino ethanol, histidine, procaine and the like. Particularly preferred are the salts of TFA and HCl.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions also can contain liquid phases in addition to and to the exclusion of water. Exemplary of such additional liquid phases are glycerin, vegetable oils such as cottonseed oil, and water-oil emulsions.

These compositions can be stored in unit or multi-dose containers, for example, sealed ampoules or vials, as an aqueous solution or as a lyophilized formulation for reconstitution. As an example of a lyophilized formulation, 10 mL vials are filled with 5 mL of sterile-filtered 1% (w/v) aqueous polypeptide solution, and the resulting mixture can then be lyophilized. The infusion solution can be prepared by reconstituting the lyophilized material using sterile Water-for-Injection (WFI).

The compositions can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

The dosage ranges for the administration of the polypeptide vary. In general, amounts are large enough to produce the desired effect in which disease symptoms of a cancer, e.g., pancreatic ductal cancer, are ameliorated. The dosage should not be so large as to cause adverse side effects. Generally, the dosage will vary with the age, condition, sex and extent of the disease in the patient and can be determined by one of skill in the art. The dosage also can be adjusted by the individual physician in the event of any complication.

A therapeutically effective amount is an amount sufficient to produce a measurable inhibition of symptoms of a condition (e.g., a reduction in tumor size or increase in subject survival time). Such symptoms are measured in conjunction with assessment of related clinical parameters.

A therapeutically effective amount of a polypeptide of this invention in the form of a polypeptide, or fragment thereof, is typically an amount of polypeptide such that when administered in a physiologically tolerable composition is sufficient to achieve a plasma concentration of from about 0.1 microgram (ug) per milliliter (mL) to about 200 ug/mL, or from about 1 ug/mL to about 150 ug/mL. In one embodiment, the plasma concentration in molarity is from about 2 micromolar (uM) to about 5 millimolar (mM) or from 100 uM to 1 mM Cthrc1 polypeptide. In other embodiments, the doses of polypeptide ranges from about 500 mg/Kg to about 1.0 g/kg (e.g., 500, 600, 700, 750, 800, 900, 1000 mg/kg).

The agents of the invention can be administered parenterally by injection or by gradual infusion over time. In other embodiments, agents are administered intravenously, intraperitoneally, intramuscularly, subcutaneously, intracavity, transdermally, topically, intraocularly, orally, intranasally, and can be delivered by peristaltic means. In one embodiment, a therapeutic compositions containing an agent of this invention are administered in a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered and timing depends on the patient to be treated, capacity of the patient's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration also are variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

Therapy

As demonstrated herein, a combination comprising a vaccine (e.g., GVAX or a Listeria monocytogenes-based vaccine that expresses ANXA2) and a PD-1 inhibitor and/or a PD-L1 inhibitor is useful for the treatment or prevention of PDA. Antibodies to various portions of PD-1 and PD-L1 can be generated using routine methods. Methods of epitope selection, antigen preparation, and antibody production are well known to those of skill in the art. PD-1 and PD-L1 antibodies are also commercially available through Bristol-Myers Squibb (NY, N.Y.), Merck (Kenilworth, N.J.), Astrazeneca (London, UK), MedImmune (Gaithersburg, Md.), Pfizer (Cambridge, Mass.), and Genentech (San Francisco, Calif.).

Therapy may be provided wherever therapy for these conditions is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of disease being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

A combination comprising a vaccine (e.g., GVAX or a Listeria monocytogenes-based vaccine that expresses ANXA2) and a PD-1 inhibitor and/or a PD-L1 inhibitor may be administered within a pharmaceutically-acceptable diluent, carrier, or excipient, in unit dosage form. Conventional pharmaceutical practice may be employed to provide suitable formulations or compositions to administer the compounds to patients suffering from a disease that is associated with a metabolic syndrome. Administration may begin before the patient is symptomatic. Any appropriate route of administration may be employed, for example, administration may be topical, parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. “Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is advantageously demonstrated at suboptimal concentrations of the compounds. Synergy can be in terms of lower cytotoxicity, increased activity, or some other beneficial effect of the combination compared with the individual components. If desired, treatment with an agent of the invention may be combined with therapies for the treatment of PDA.

Kits

The invention provides kits for the treatment or prevention of a PDA. In one embodiment, the kit includes a therapeutic or prophylactic composition containing an effective amount of an agent described herein. In some embodiments, the kit comprises a sterile container that contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired an agent of the invention is provided together with instructions for administering the agent to a subject having or at risk of developing a cancer. The instructions will generally include information about the use of the composition for the treatment or prevention of a cancer. In other embodiments, the instructions include at least one of the following: description of the therapeutic agent; dosage schedule and administration for treatment or prevention of a cancer or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

This invention is further illustrated by the following examples, which should not be construed as limiting. All documents mentioned herein are incorporated herein by reference.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLE 1

### Materials and Methods

Study Subjects and Tissue Specimens

Tumor tissues for immunohistochemistry staining (IHC) were obtained from specimens collected after vaccine exposure and unvaccinated patients who underwent surgery concurrently. Formalin-fixed paraffin-embedded tissue blocks were obtained from a pathology archive. In most cases, a part of the tumor was also stored at −80° C. in OCT freezing medium.

Cell Lines and Medium

Panc02 is a highly tumorigenic cell line derived from methylcholanthrene treated C57B16 mice (Corbett et al., 1984 Cancer Research, 44(2):717-26; Leao et al., 2008 Clinical and Translational Science, 1(3):228-39). Panc02 cells were maintained in DMEM media (Life Technologies, Frederick, Md., USA), 10% Fetalclone II (ThermoScientific, Rockville, Md., USA), 1% L-glutamine (Life Technologies, Frederick, Md., USA), and 0.5% Penicillin/Streptomycin (Life Technologies, Frederick, Md., USA) at 37° C. in 10% CO₂. B78H1 cells are an MHC class I-negative variant of B 16 melanoma cell line engineered to secrete GM-CSF (Leao et al., 2008 Clinical and Translational Science, 1(3):228-39, incorporated herein by reference; Levitsky et al., 1994 The Journal of Experimental Medicine, 179(4):1215-24, incorporated herein by reference). B78H1 cells were maintained in RPMI media (Life Technologies, Frederick, Md., USA), 10% Fetalclone II, 0.5% L-glutamine, and 1% Penicillin/Streptomycin at 37° C. in 5% CO₂. Immune analysis was performed using CTL medium which consisted of RPMI media, 10% fetal bovine serum (Atlas Biologicals, Fort Collins, Colo., USA), 1% L-glutamine, 0.5% Penicillin/Streptomycin, and 0.1% 2-mercaptoethanol (Life Technologies, Frederick, Md., USA).

Human PDA PD-L1 Immunohistochemistry

Human PD-L1 IHC staining of paraffin embedded pancreatic tumor specimens was done using the Dako Catalyzed Signal Amplification system as previously described (Bigelow et al., 2013 Journal of Visualized Experiments: JoVE, 71). A PDA is considered to be positive for PD-L1 expression if membranous staining is present in more than 5% of the neoplastic cells in the PDA, as previously described (Brahmer et al., 2010 Journal of clinical oncology: official journal of the American Society of Clinical Oncology, 28(19):3167-75; Taube et al., 2012 Science Translational Medicine, 4(127):127ra37).

PD-L1 Murine Immunofluorescence Staining

After hemispleen injection, murine liver necropsies were performed. Liver tissue (frozen in OCT at −80° C.) was cryo-cut for slides. At the time of staining, slides were thawed, fixed in 4% PFA for 5 min and subsequently washed in TBS with 0.1% tween (TBST) for 5 minutes three separate times. Slides were then blocked with 10% goat serum in PBS for 30 minutes followed by a repeat wash as above. Primary rat anti-mouse B7-H1 antibody (MIH5, eBioscience) and rat anti-mouse IgG 2ak (R35-95, BD Pharmingen) at 1:50 dilution was added for 60 minutes. Slides were washed as above and secondary goat anti-rat IgG-FITC antibody (Southern Biotech) at 1:200 dilution was added for 30 minutes. Slides were washed with TBST for 10 minutes three times and mounted with Vectashield Dapi containing kit.

Mice and In Vivo Experiments

Six to eight week old C57B16 female mice were purchased from Harlan Laboratories (Frederick, Md., USA). Tumor inoculation was performed via hemispleen technique on day 0 as previously described (Jain et al., 2003 Annals of Surgical Oncology, 10(7):810-20; Olino et al., 2012 Annals of Surgical Oncology, 19 Suppl 3:S597-607; Soares et al, JoVE 2013, in press). Briefly, the spleen was eviscerated from the anesthetized mouse, clipped and divided in half. One half of the spleen is injected with 2×10⁶ Panc02 tumor cells. The injected hemispleen is subsequently removed. On day 3, a single dose of cyclophosphamide (Cy) (100 mg/kg) was administered intraperitoneally (IP). Hamster anti-mouse PD-1 G4 antibodies (100 μg IP), hamster anti-mouse PD-L1 10B5 (100 μg IP), and hamster IgG control (100 μg IP) (Rockland Immunochemicals Inc, Boyertown, Pa., USA) was administered on day 3 and twice weekly until death.

Murine GVAX vaccine was formulated as previously described (Leao et al., 2008 Clinical and Translational Science, 1(3):228-39, incorporated herein by reference). Specifically, whole-cell Panc02 cells (antigen source) was mixed with the bystander GM-CSF-secreting B78H1 cell line (GM-CSF source) as the vaccine. Vaccine cells were washed in PBS, irradiated at 50 Gy and administered subcutaneously in three limbs (0.1 mL) on days 4, 7, 14, and 21. Mice were monitored three times per week for survival analysis and euthanized by CO₂ inhalation following IACUC approved criteria or at study endpoint of 90 days.

Analysis of Spleen and Liver Infiltrating Lymphocytes

On day 13 following hemispleen injection, murine livers and spleens were removed. For analysis of CD4⁺ and CD8⁺ T cells, livers and spleens were processed either individually or pooled as a group. Each liver was mashed through 100-μm and 40-μm nylon filter and brought to a volume of 25 mL CTL medium. Each spleen was mashed through 100-μm nylon filter and brought to a volume of 15 mL CTL medium. All suspensions were centrifuged at 1500 rpm for 5 minutes. Liver cell pellets were then suspended in 5 mL 80% Percoll (GE Healthcare Life Sciences, Pittsburgh, Pa.), overlaid with 5 mL 40% Percoll, and centrifuged at room temperature for 25 minutes at 3200 rpm, without brake. The lymphocyte layer was removed and suspended in 10 mL CTL media.

Cell Staining and Flow Cytometry

Following the isolation of spleen and liver-infiltrating lymphocytes from murine livers and hemispleens, cells were stained with Live Dead Near-IR Dead Cell kit (Invitrogen), CD3-APC (Biolegend), CD3-APC-Cy7 (Biolegend), CD8-PeCy7 (Biolegend), CD4-V500 (BD Horizon), CD25-BV421 (Biolegend), CTLA-4-BV421 (Biolegend), and CD69-FITC (BD Pharmingen) for 30 minutes and assayed on an LSR II flow cytometer (BD Biosciences).

Intracellular Staining for Foxp3 and Flow Cytometry

After staining for CD4 and CD25 following the above protocol, isolated liver infiltrating lymphocytes and splenocytes were suspended in cold Fix/Perm buffer (eBioscience) and incubated for 30 minutes at 4° C. The cells were then washed with Perm Buffer (eBioscience) and blocked with mouse Fc antibody (BD Pharmingen) for 15 minutes. Anti-mouse forkhead box P3 (FoxP3)-AF488 (MF23; BD Pharmingen) antibody was added and incubated at 4° C. for 30 min. Cells were washed and assayed on an LSR II flow cytometer.

Intracellular Cell Staining for IFNγ and Flow Cytometry

Isolated liver-infiltrating lymphocytes and splenocytes were enriched for CD8 cells using CD8 negative isolation kits (Life Technologies) according to manufacturer's protocol. CD3:CD28 stimulation beads (Life Technologies) were added to isolated CD8+ T cells and incubated for 12 hours at 37° C. in 5% CO2 according manufacturer's protocol. Golgistop (1:1000; BD Biosciences) was added and incubated for 5 hours at 37° C. in 5% CO₂. After removing the beads according to manufacturer's protocol and washing the cells twice with flow buffer, cells were stained with CD8, CD3, and live dead Near-IR stain according to the previously mentioned protocol. The cells were then washed twice, suspended in cytofix/cytoperm buffer (BD Biosciences), incubated at 41° C. for 30 minutes, and then washed with Permwash (BD Biosciences). IFNγ-BV421 (Biolegend) antibody was added in Permwash and incubated at 41 C for 20 minutes. Flow cytometry assays were completed on an LSR II flow cytometer.

Mouse IFNc Enzyme-Linked Immunosorbent Assay

Isolated liver-infiltrating lymphocytes and splenocytes were enriched for CD8 cells using CD8 negative isolation kits (Life Technologies) according to manufacturer's protocol. Irradiated Panc02 tumor cells were added to isolated CD8+ T cells at a ratio of 5:1 (2×10⁵ CD8+ T cells with 4×10⁴ Panc02 tumor cells) and incubated for 18 hours at 37° C. Mouse IFNγ ELISA Ready-SET-Go assay was conducted per manufacturer protocol (eBioscience).

Statistical Analysis.

Statistical analyses for survival were conducted using Kaplan-Meier curves and log-rank test for survival. For comparison of cure rates the values were evaluated using χ² test. For comparison of cell number, percentage, and cytokine expression between 2 groups, the mean values were evaluated using unpaired Student t test. P<0.05 was considered statistically significant. For comparison of cell infiltration and cytokine expression, the mean values were evaluated using unpaired student's t-test. P≦0.05 was considered statistically significant.

### EXAMPLE 2

### PD-L1 Expression is Upregulated Following GVAX Administration when Compared with Untreated Human and Mouse PDA Tumors

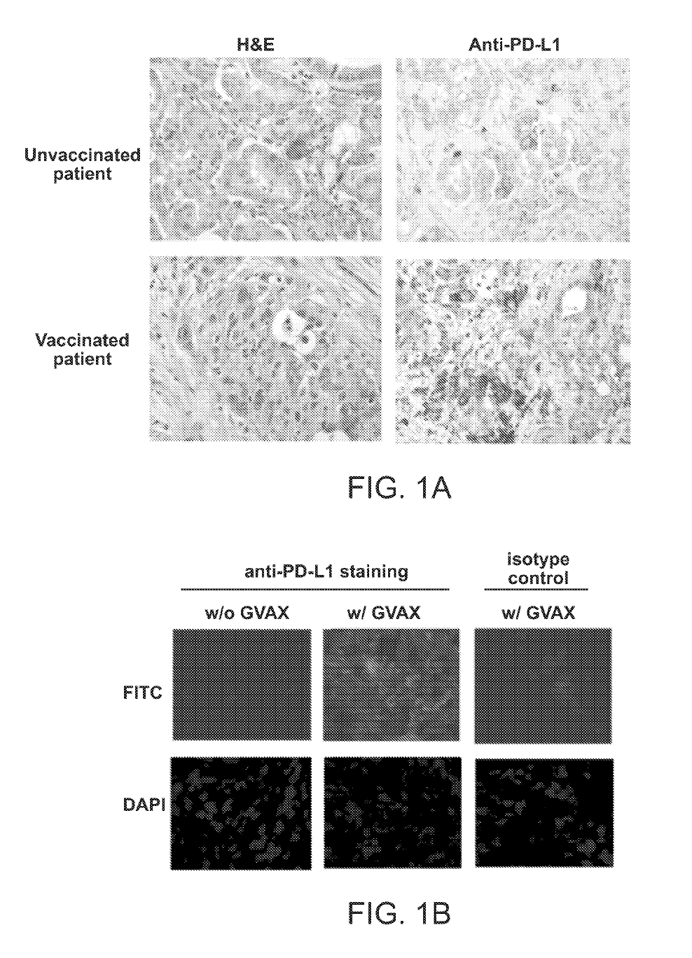

To study the role of PD-L1/PD-1 signaling in regulating antitumor immune responses in PDA, PDL1 expression in the neoplastic cells of surgically resected PDA was examined Specifically, PDAs resected from 25 patients who underwent pancreaticoduodenectomies were examined. Similar to how the PD-L1 expression was characterized in melanoma (Brahmer et al. J Clin Oncol. 2010; 28: 3167-3175; Taube et al. Sci Transl Med. 2012; 4:127ra37) a PDA was considered to be positive for PD-L1 expression if membranous staining was present in >5% of the neoplastic cells in the PDA. IHC analysis revealed that approximately 12.5% (3 of 25 analyzed) of resected PDAs from unvaccinated patients were positive for PD-L1 expression based on this previously published criteria and, that the intensity of the membranous staining of PD-L1 in these PDAs was also weak (FIG. 1A). Next, the PD-L1 membranous expression in PDAs from patients who received the GVAX vaccine 2 weeks before surgical resection in the aforementioned clinical trial was examined (Lutz et al. Cancer Immunol Res. 2014; 2:616-631). An increased intensity of PD-L1 membranous staining was identified on the epithelial tumor cells of PDAs from these vaccinated patients when compared with those from unvaccinated patients. The frequency of PDAs considered positive for PD-L1 membranous expression was moderately increased to 25% (10 of 40 analyzed) in vaccinated patients and strong PD-L1+ signals were observed in all the vaccine-induced intratumoral tertiary lymphoid aggregates found in the majority (>80%) of PDAs from vaccinated patients (Lutz et al. Cancer Immunol Res. 2014; 2:616-631).

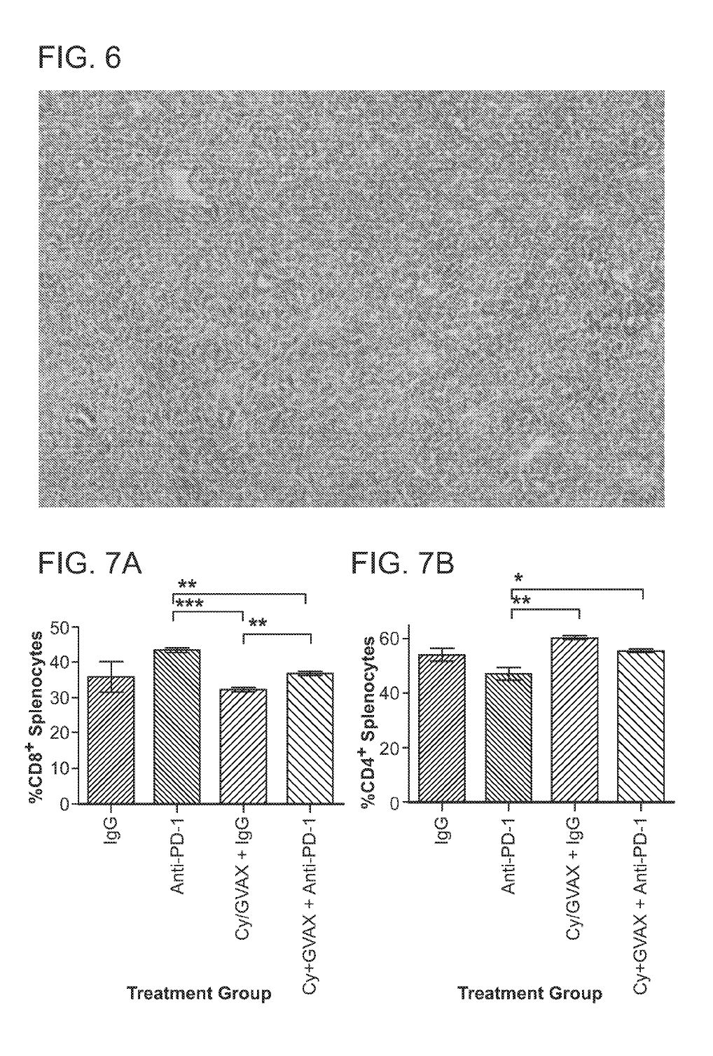

To better understand the significance of PD-1/PD-L1 regulation of immune responses within the PDA TME, it was next examined whether GVAX therapy can also induce the upregulation of PD-L1 expression in a preclinical model of metastatic PDA. A previously reported experimental model of liver metastases was utilized in which Panc02 tumor cells were injected directly into the spleen. A hemisplenectomy was performed to remove residual tumor cells and to allow the establishment of liver metastases where all untreated mice die from the development of diffuse liver metastases within 6 weeks (FIG. 6) (Soares et al., J Vis Exp. 2014; 91:e51677; Zheng L, Jaffee E M. Oncoimmunology. 2012; 1:112-114). Metastasis-bearing mice were treated with GVAX 4 and 7 days after hemispleen injection and harvested the liver 2 weeks after tumor inoculation to perform immunofluorescence staining for PD-L1 expression. Similar to the findings in human PDAs, livers from untreated mice receiving no treatment had no evidence of PD-L1 expression, whereas livers from GVAX-treated mice had significant induction of PD-L1 membranous expression (FIG. 1B). The addition of αPD-1 antibody to GVAX therapy did not alter PD-L1 expression in murine liver metastases when compared with GVAX monotherapy. Thus, these data demonstrate that similar to human PDA following GVAX treatment (FIG. 1A), GVAX is also able to induce PD-L1 expression in murine PDAs.

### EXAMPLE 3

### Combination Therapy with GVAX and PD-1 or PD-L1 Blockade Improves Survival in a PDA Mouse Model

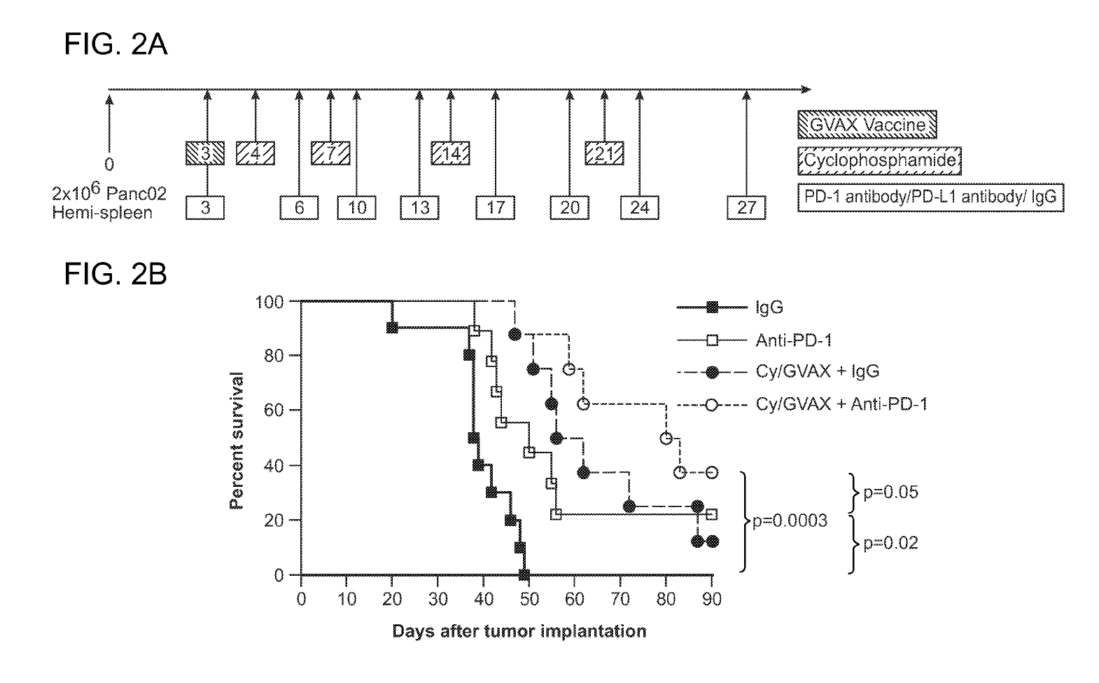

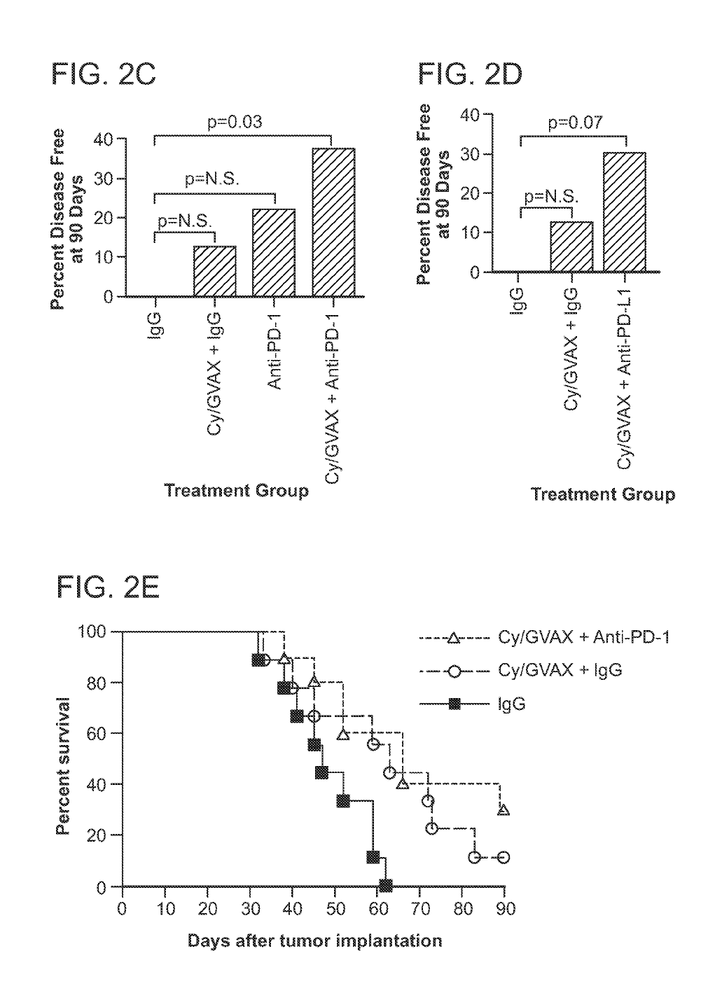

Next, it was examined whether blocking PD-L1, or its receptor PD-1, can augment the antitumor activity of GVAX in the PDA hemisplenectomy model. GVAX was administered on days 4, 7, 14, and 21 (FIG. 2A). A single low dose of Cy was given on day 3 for Treg depletion as reported for other GVAX preclinical models (Machiels et al. Cancer Res. 2001; 61:3689-3697; Wada et al. Cancer Res. 2009; 69:4309-4318; Radojcic et al., Cancer Immunol Immunother. 2010; 59: 137-148) and hamster anti-mouse PD-1, PD-L1 monoclonal antibodies, or IgG control were administered on day 3 as either monotherapy or in combination with Cy/GVAX.

Although both αPD-1 monotherapy [median overall survival (OS), 50 d] and Cy/GVAX therapy alone (OS, 59 d) improved the survival of mice compared with IgG control treatment (OS, 38.5 d; P<0.05), Cy/GVAX+αPD-1 combination therapy significantly increased median survival compared with αPD-1 monotherapy (OS, 81.5 vs. 50 d; P=0.05) (FIG. 2B). A trend toward improved survival was seen with Cy/GVAX+αPD-1 combination therapy compared with Cy/GVAX therapy alone (OS, 81.5 vs. 59 d; P=0.22). Moreover, the combination therapy cured a larger percentage of mice (38%) (FIG. 2C) when compared with Cy/ GVAX (12.5%) therapy or αPD-1 monotherapy (22%). Similar experiments were performed to investigate the Cy/GVAX+αPD-L1 combination therapy. This combination cured 30% of mice (FIGS. 2D, E), compared with an 11% cure rate with Cy/GVAX therapy alone. These data indicate that PD-1 or PD-L1 blockade therapy enhances the antitumor activity of Cy/GVAX.

### EXAMPLE 4

### GVAX Combined with PD-1 Blockade Increases CD8⁺ T Lymphocytes in PDAs

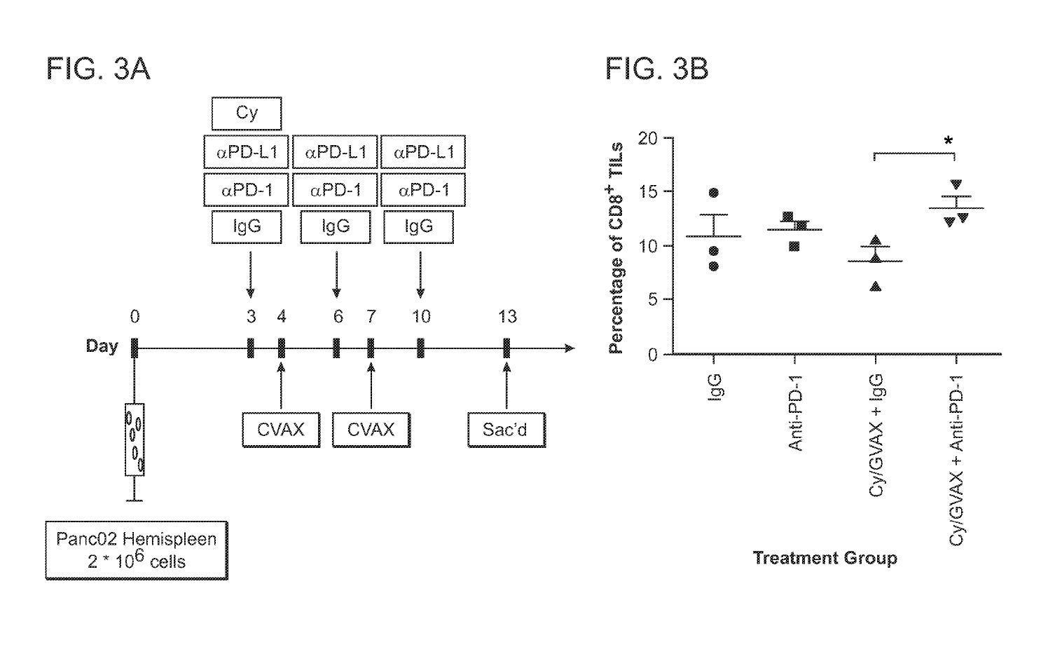

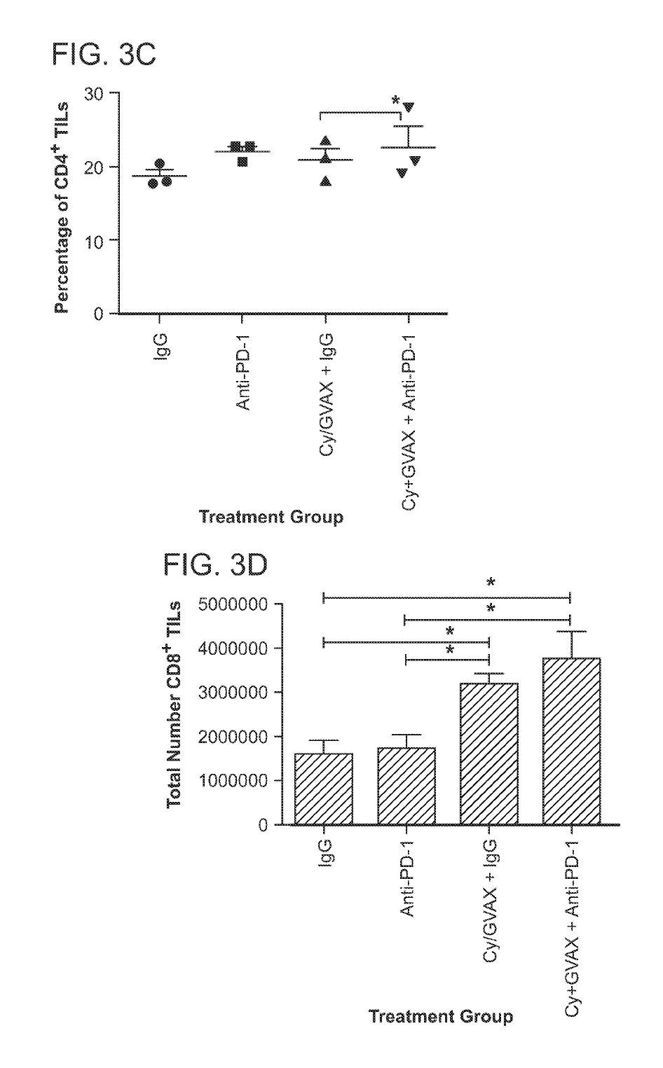

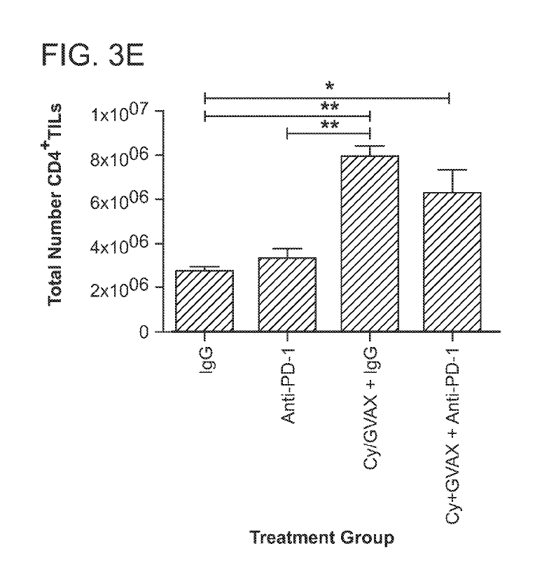

To define the immune mechanisms by which PD-1 or PD-L1 blockade enhances the antitumor activity of Cy/GVAX, the effect of each single immunotherapy and combined treatment on the composition of T lymphocytes infiltrating the metastatic PDA TME was evaluated. Tumor-bearing mice were treated with either aPD-1 or IgG control. Cy was administrated on day 3 and GVAX was administered twice on days 4 and 7 (FIG. 3A). On day 13, livers and spleen were harvested for fluorescence-activated cell sorting analysis of splenocytes and tumor infiltrating lymphocytes (TILs) in liver.

TIL numbers were increased in the livers of mice treated with Cy/GVAX+αPD-1 combination therapy where a statistically significant and approximately 60%increase in the percentage of CD8+ T cells among lymphocytes infiltrating the TME was seen in mice treated by the combination when compared with Cy/GVAX alone (13.4% vs. 8.57%, P=0.04) (FIG. 3B). By contrast, there was no significant change in CD4+ T cells in the TILs of mice treated with combination therapy compared with Cy/GVAX alone (22.6% vs. 20.9%) (FIG. 3C). It is interesting to note that Cy/GVAX alone significantly increased the absolute numbers of CD8+ and CD4+ TILs, but not the percentage of CD8+ and CD4+ T cells among TILs, compared with no treatment controls (FIGS. 3D, E). This result suggests that other lymphocyte subtypes were also increased in the TILs following the Cy/GVAX treatment. It should be noted that the Cy/GVAX+ αPD-1 combination significantly increases the number of CD8+ TILs per mouse compared with αPD-1 alone but not to Cy/GVAX alone, suggesting that addition of αPD-1 to Cy/GVAX mainly changes the T-cell composition in the TILs. Systemically, mice treated with combination therapy had less of an increase in CD8+ T-cell composition in their splenocytes compared with those treated with the Cy/GVAX alone (36.8% vs. 32.5%, P<0.01) (FIG. 7A-FIG. 7B). These data indicate that αPD-1 therapy enhances the antitumor activity of Cy/GVAX by selectively increasing the composition of CD8+ T cells in the TME.

### EXAMPLE 5

### Cy/GVAX Combined with PD-1 Blockade Enhances the Activation of Tumor-Specific IFNc Production in CD8+ T Cells within the Metastatic PDA TME

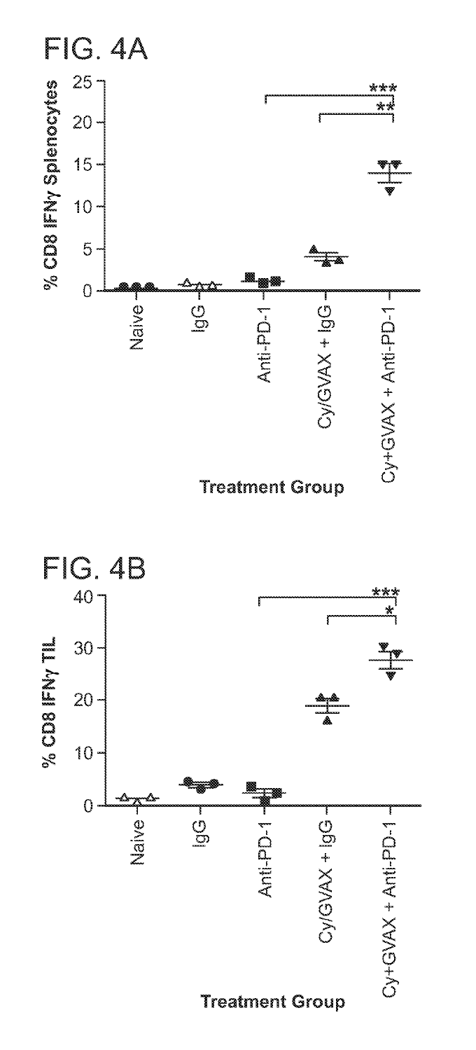

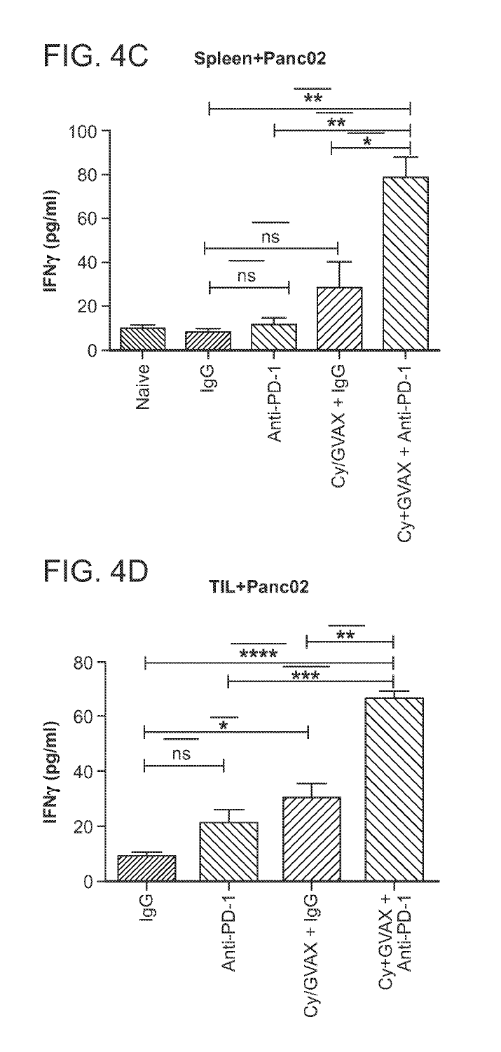

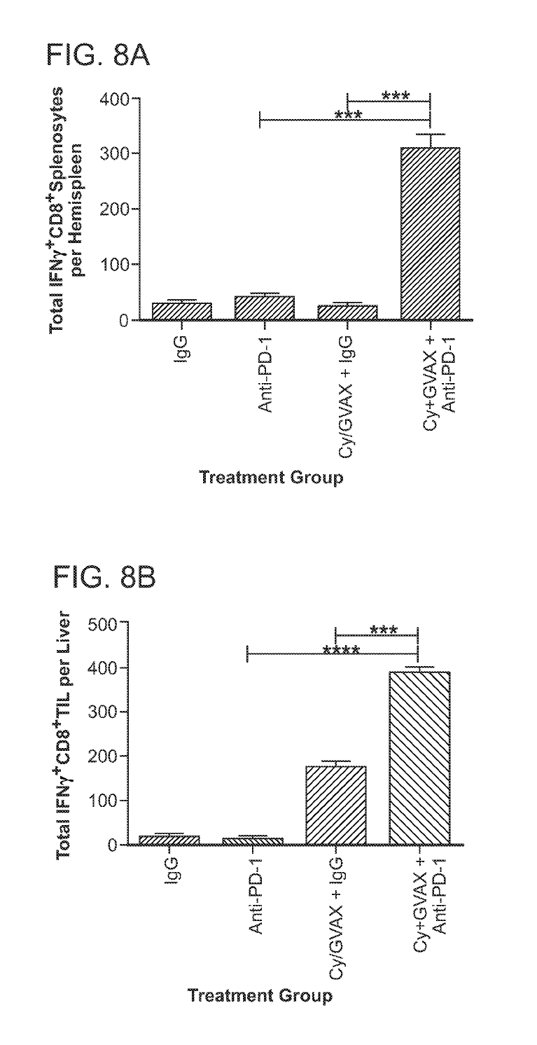

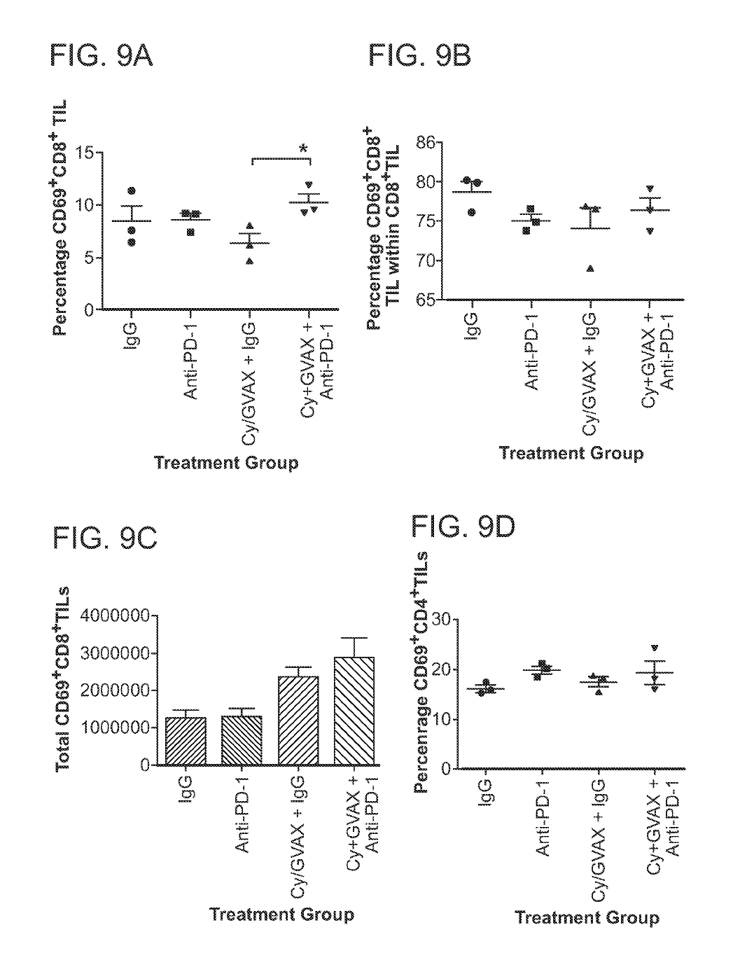

To determine whether PD-1 blockade enhances T-cell activation in the TME, IFNγ production by CD8+ T cells was examined in splenocytes and TILs. There were significantly greater numbers of IFNγ-producing CD8+ T cells in the spleens of mice treated with Cy/GVAX+αPD-1 combination versus Cy/GVAX alone (13.9% vs. 4%, P<0.01) or αPD-1 monotherapy (13.9% vs. 1.1%, P<0.001) (FIG. 4A). In addition, the Cy/GVAX+αPD-1 combination resulted in a significant increase in the percentage of IFNγ-producing CD8+ T cells within TILs when compared with αPD-1 monotherapy (27.6% vs. 2.3%, P<0.001) or Cy/GVAX alone (27.6% vs. 18.9%, P<0.05) (FIG. 4B). The total number of IFNγ-producing CD8+ T cells in splenocytes (FIG. 8A) and in TILs (FIG. 8B) were also significantly increased with the combinational therapy compared with Cy/GVAX or αPD-1 alone. Similar results were observed when αPD-L1 blockade was used instead of αPD-1 blockade. Although there was an increase of CD69+CD8+ cells in the TILs from mice treated with Cy/GVAX+αPD-1 combination compared with either monotherapy, there was not an increase in the percentage of CD69+-activated cells among CD8+ TILs (FIG. 9A-FIG. 9C). These results suggest that the addition of αPD-1 to Cy/GVAX does not further activate CD8+ TILs, but may have increased its IFNγ-mediated cytotoxic activity.

Next, tumor-specific CD8+ T-cell activity was assessed both systemically and within the TME with the murine IFNγ ELISA analysis by using irradiated autologous tumor cells as a target. Compared with IgG controls, neither Cy/GVAX nor αPD-1 alone significantly enhanced the tumor-specific IFNγ secretion by CD8+ T cells in splenocytes. In contrast, Cy/GVAX alone enhanced the tumor-specific IFNγ secretion by CD8+ T cells in the TME when compared with αPD-1 alone Importantly, mice treated by Cy/GVAX+αPD-1 combination therapy demonstrated significantly greater IFNγ secretion by CD8+ T cells compared with either Cy/GVAX or αPD-1 alone in both splenocytes (FIG. 4C) and TILs (FIG. 4D) in response to Panc02 tumor cells.

### EXAMPLE 6

### Cy/GVAX and αPD-1 Combination Overcomes Treg, CTLA-4, and PD-1/PD-L1 Immunosuppressive Pathways

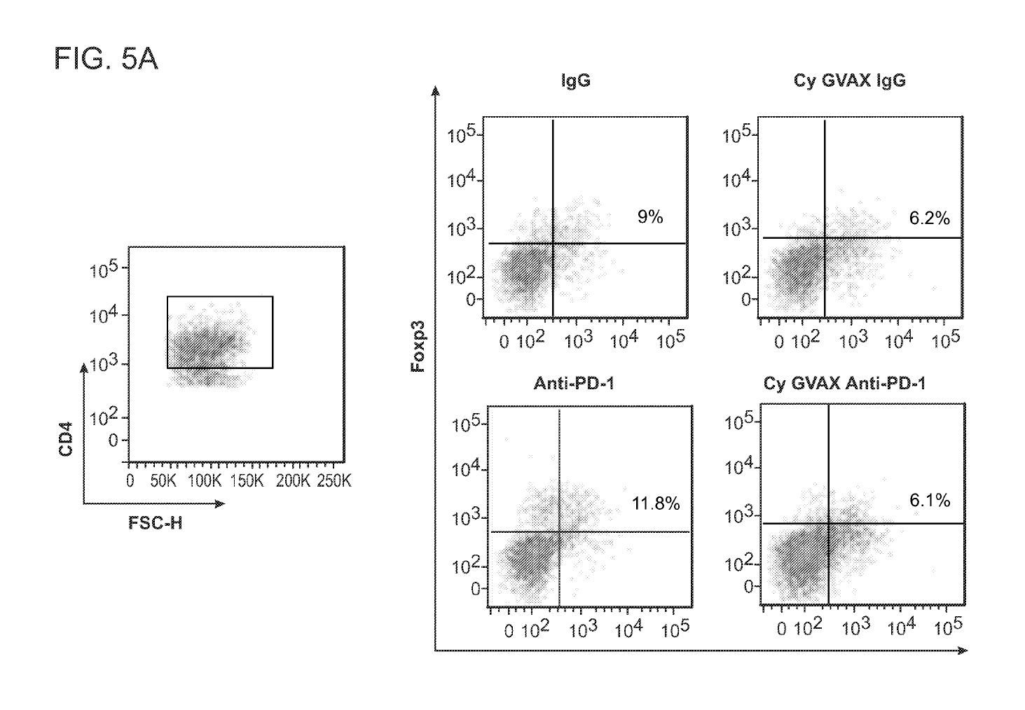

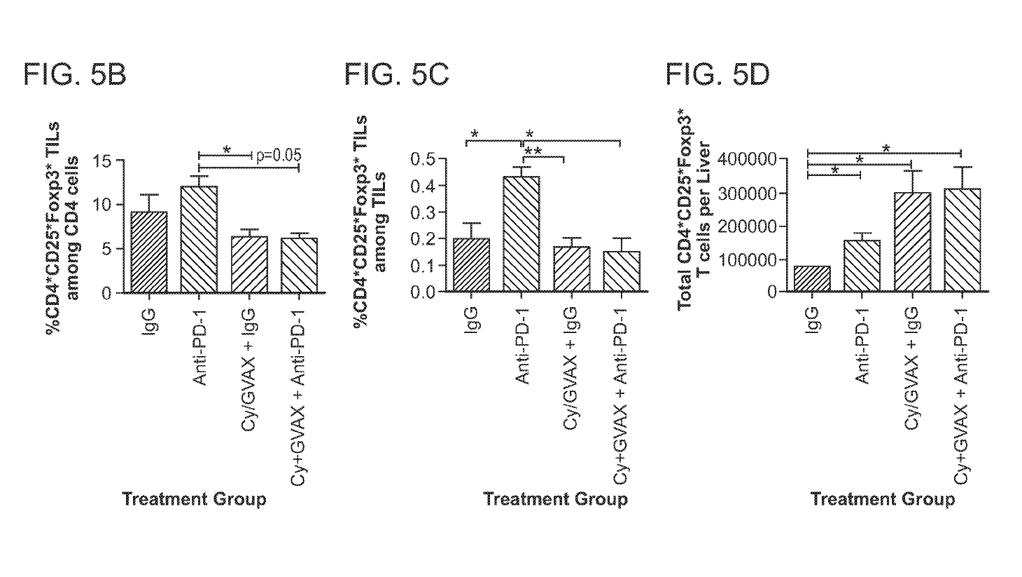

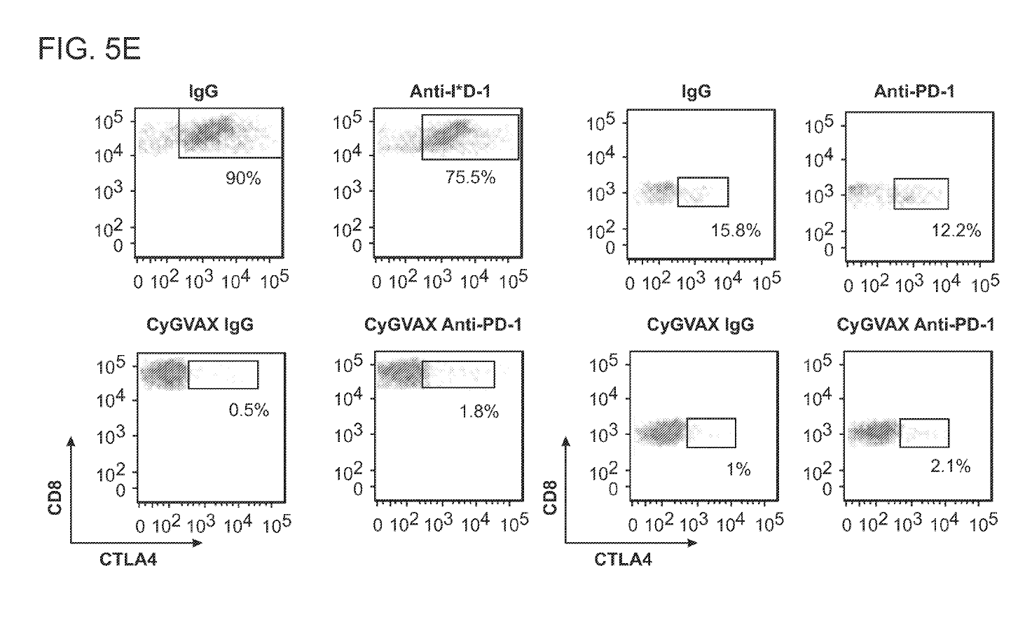

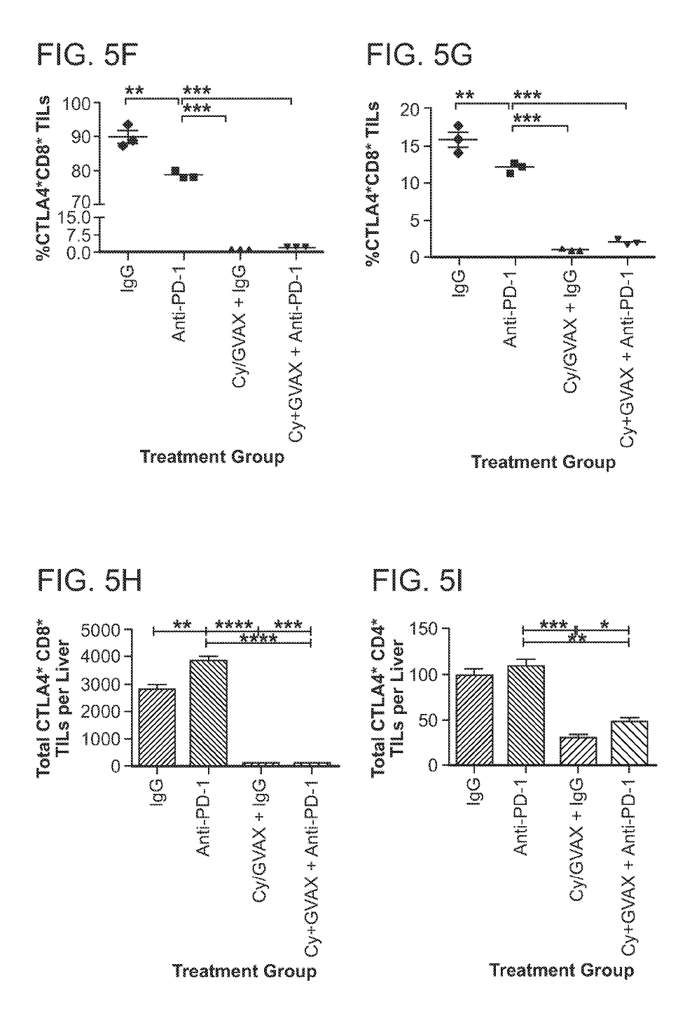

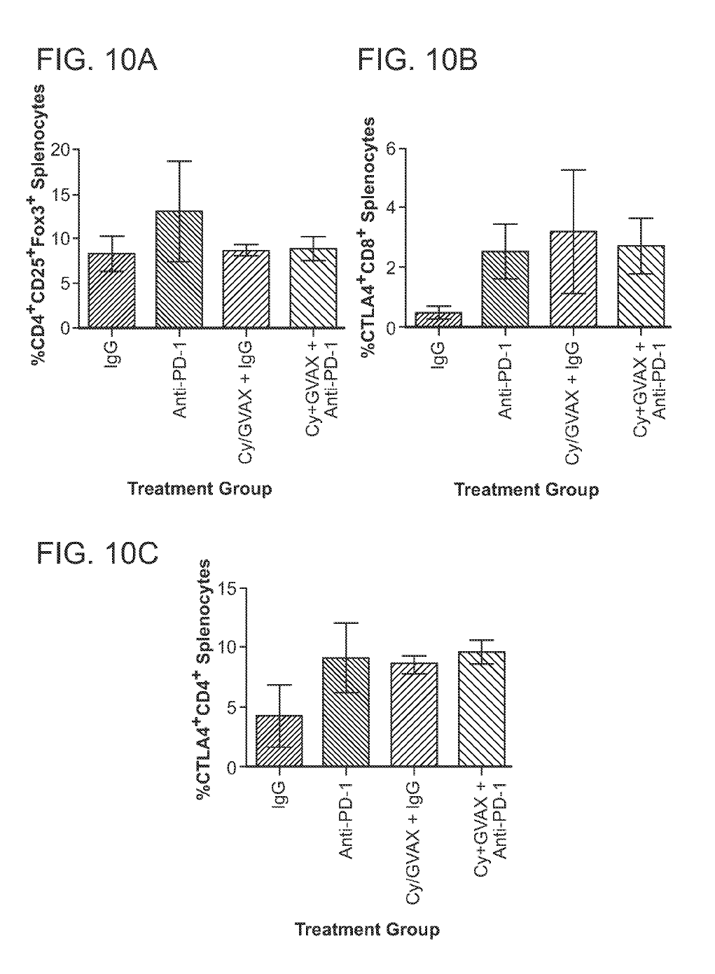

The effect of PD-1/PD-L1 blockade on immune activation is likely the result of blocking the PD-L1/PD-1 immune checkpoint pathway. However, other checkpoint pathways may also be involved. Thus, the effect of αPD-1 blockade therapy on Treg population and CTLA-4 expression levels in splenocytes and TILs was determined. Anti-PD-1 monotherapy significantly increased the percentage of CD4+CD25+Foxp3+ Tregs among TILs (FIG. 5A-FIG. 5C) and modestly increased that in splenocytes (FIG. 10A) when compared with Cy/GVAX or IgG alone. The addition of Cy/GVAX to αPD-1 blockade resulted in a decrease in the percentage of Treg among CD4+ TILs in comparison with αPD-1 monotherapy. It is interesting to note that increasing total numbers of Tregs within the TME were seen in mice treated by αPD-1 alone, Cy/GVAX alone, or Cy/GVAX+αPD-1 combination therapy comparing to mice treated by IgG alone (FIG. 5D). Although it cannot be excluded that FOXP3 is upregulated by PD-1 blockade in effector T cells without acquiring suppressive activity, the results suggest another possibility that Cy/GVAX abrogates the effect of PD-1 blockade on the percentage of CD4+CD25+Foxp3+ among TILs.

Anti-PD-1 monotherapy significantly decreased CTLA-4+CD4+ and CTLA-4+CD8+ T cells in the TME compared with IgG controls. However, Cy/GVAX alone had a greater effect on decreasing CTLA-4 expression and the addition of Cy/GVAX to αPD-1 blockade therapy significantly decreases both CTLA-4+CD4+ and CTLA-4+CD8+ T cells compared with αPD-1 monotherapy (FIGS. 5E-5I). Notably, the addition of Cy/GVAX to αPD-1 therapy did not significantly decrease CTLA-4+ T cells in the spleen (FIGS. 10B and 10C). Taken together, these data indicate that Cy/GVAX and αPD-1 antibodies cooperate to overcome multiple immunosuppressive pathways including Tregs, CTLA-4, and PD-L1/PD-1 signaling.

As described herein, PD-L1 expression is upregulated in both human and murine PDAs when IFNγ-producing CD8+ T cells infiltrate the TME after Cy/GVAX therapy, supporting for the first time that the process of adaptive resistance can occur in PDA. It was further demonstrated that PD-1 blockade therapy overcomes this vaccine-induced adaptive resistance and enhances vaccine-induced effector T-cell response in mouse PDA.

Delayed tumor growth in mouse models of PDA with αPD-L1 monotherapy has been reported; however, αPD-L1 single-agent therapy did not eradicate tumors that were either implanted subcutaneously or orthotopically in the pancreas (Nomi et al. Clin Cancer Res, 2007; 13:2151-2157; Okudaira et al. Int J Oncol. 2009; 35:741-749). Described herein is an effect of αPD-1 monotherapy in prolonging survival and curing a small percentage of metastasis-bearing mice. This modest effect of αPD-1 monotherapy in the model described herein may be exerted on the PD-L1/PD-1 pathway mediated by PD-L1-expressing monocytes or antigen-presenting cells that have not yet been examined herein (Selenko-Gebauer Net al. J Immunol, 2003; 170:3637-3644). However, overall survival and cure rates were significantly improved when Cy/GVAX was combined with αPD-1/PD-L1 blockade. As such, as described herein, it was determined whether a T-cell augmenting agent that increases IFNα-expressing T cells in the PDA TME should be given with αPD-1/αPD-L1 blockade to achieve a significant clinical response in patients with PDA.

The additive benefit of combining vaccine therapy with PD-1 or PD-L1 blockade has been identified in other tumor models (Duraiswamy et al., Cancer Res. 2013; 73:6900-6912; Duraiswamy et al., Cancer Res. 2013; 73:3591-3603; Li et al. Clin Cancer Res., 2009; 15:1623-1634; Mkrtichyan et al. Eur J Immunol, 2011; 41:2977-2986). However, in both CT26 colorectal tumor and ID8 ovarian tumor subcutaneous models, αPD-1/αPD-L1 or αCTLA-4 monotherapy appeared to result in significantly more enhanced effector T-cell immune responses and antitumor activity than αPD-1/αPD-L1 did in the liver metastasis model of PDA described herein (Duraiswamy et al. Cancer Res, 2013; 73:3591-3603). In addition, it was observed that αPD-1/αPD-L1 monotherapy increased the percentage of Tregs in the lymphocytes infiltrating the tumors formed by Panc02 cells, whereas others have shown that αPD-1/αPD-L1 monotherapy reduces Tregs in tumors formed by CT26 or ID8 cells (Duraiswamy et al. Cancer Res, 2013; 73:3591-3603). The differences in treatment response are likely attributed to the difference in the tumor types. Both αPD-1/αPD-L1 monotherapies were associated with objective responses in colorectal and ovarian cancer patients, but were not effective in metastatic PDA patients in clinical trials (Brahmer et al. N Engl J Med, 2012; 366:2455-2465; Topalian et al. N Engl J Med. 2012; 366:2443-2454). In addition, the liver metastasis model is a more physiologically relevant model than subcutaneous models as the liver is the most common site of metastasis for PDA and the majority of PDA patients have metastatic disease at the time of diagnosis.

As described above, αPD-1 monotherapy increased the percentage of Tregs in lymphocytes infiltrating the TME. A a similar increase of Tregs was identified in the TME when GVAX is given without Cy. Moreover, the addition of Cy to GVAX therapy has been previously shown to induce the recruitment of high avidity CD8+ T cells which was attributed to Treg depletion (Ercolini et al. J Exp Med. 2005; 201:1591-1602). Previous reports have studied the effectiveness of GVAX in the treatment of murine colorectal cancer hepatic metastases and demonstrated that Cy in conjunction with CT26 GVAX resulted in transient depletion of Tregs as well as expansion of tumor antigen-specific T cells (Jain et al. Ann Surg Oncol, 2003; 10:810-820; Radojcic et al. Cancer Immunol Immunother, 2010; 59: 137-148). Described herein are results that show that Cy/GVAX had a more significant effect in suppressing CTLA-4+ T cells than αPD-1 blockade. The addition of Cy/GVAX to αPD-1 therapy abrogates the αPD-1-induced upregulation of Tregs and significantly downregulates CTLA-4 expression in CD4+ and CD8+ T cells. The role of Cy with or without GVAX and the role of GVAX with or without Cy was not described herein. However, an enhancement in antitumor activity was not identified with PD-1/PD-L1 blockade and a single intraperitoneal dose of Cy alone, or with GVAX alone.

In conclusion, the results presented herein provide evidence of therapy-induced adaptive resistance with induction of PD-L1 expression in a murine PDA. It supports the addition of αPD-1 or αPD-L1 blockade to Cy/GVAX-based immunotherapy to achieve durable tumor responses. In some cases, adding Cy/GVAX therapy to αPD-1 or αPD-L1 therapy also overcomes additional immune checkpoint mechanisms. As such, the Cy/GVAX and PD-1 or PD-L1 blockade combination therapy is examined in pancreatic cancer patients.

### EXAMPLE 7

### Sequential Treatment with a Listeria-Based Vaccine and PD-1 Blockade Antibody Improves Survival in a Murine Model of Pancreatic Ductal Adenocarcinoma

As described in detail above, pancreatic ductal adenocarcinoma is characterized by a highly immunosuppressive tumor microenvironment (TME). As described above, a neoadjuvant study was designed to evaluate post-immunotherapy changes within TME following treatment with the GM-CSF-secreting whole cell vaccine (GVAX). It was found that vaccine therapy, by inducing PD-L1 expression in TME, primed PDA for anti-PD-1/PD-L1 therapies. Subsequently, as described above, it was demonstrated that anti-PD-1/PD-L1 therapies enhanced the anti-tumor activity of GVAX in the preclinical model of PDAC.

Annexin A2 (ANXA2) was also identified as an antigen targeted by GVAX. As described below, a Listeria monocytogenes (Lm)-based vaccine (Lm-ANXA2) was developed that specifically expresses the ANXA2 antigen.

Live attenuated Listeria monocytogenes (LM) is a bacterial vector able to induce a T-cell response to tumor-associated antigens and is useful in vaccine development (Chen et al., 2012 Oncogene, 31(17): 2140-2152, incorporated herein by reference). Live-attenuated Listeria monocytogenes naturally targets dendritic cells in vivo and stimulates both innate and adaptive cellular immunity. Lm-based vaccines engineered to express cancer antigens have demonstrated striking efficacy in several animal models and have resulted in encouraging anecdotal survival benefit in early human clinical trials. Lm strains have been modified to decrease virulence while maintaining immunogenicity (Le et al., 2012 Semin Oncol, 39(3): 311-322, incorporated herein by reference).

Annexin 2 (ANXA2) refers in particular to Homo sapiens Annexin 2. Human Annexin 2 mRNA is set forth in GenBank Accession No. BC093056 (BC093056.1), incorporated by reference herein in its entirety. Human ANXA2 protein is provided in Genbank Accession No. AAH93056 (AAH93056.1), incorporated herein by reference it its entirety. ANXA2 is a involved in tumor metastases and is a potential antigenic target for cancer immunotherapy (Foley, K. et al. PLoS ONE 2011; 6(4): e19390.; Jaffee, E M. OncoImmunology 2012; 1(1):112-114.) The ANXA2 antigen is described in Zheng L et al., 2011 PLoS ONE 6(4): e19390 and Zheng L and Jaffee E M 2012 OncoImmunology, 1(1): 112-114, each of which is incorporated herein by reference.

To test whether anti-PD-1 therapies can enhance the anti-tumor activity of non-GVAX-based vaccines, syngeneic mice implanted with murine PDA cells were treated with 1) empty Lm vaccine followed sequentially by (>) IgG, 2) empty Lm>anti-PD-1 antibody, 3) Lm-AnxA2>IgG, or 4) Lm-AnxA2>anti-PD-1 antibody.

Unexpectedly, as shown in Table 1, Lm-AnxA2 significantly prolonged the survival of PDA tumor-bearing mice compared to empty Lm (44 vs. 29 days; p=0.026). Median survival was not prolonged by adding anti-PD-1 antibody to either Lm AnxA2 or empty Lm vaccine. Nevertheless, the percentage of disease-free mice on Day 75 increased from 18% to 45% with the addition of anti-PD-1, thereby supporting a survival benefit from the combination.

The AnxA2 specific listeria vaccine demonstrated antitumor activity in the preclinical model of PDA. The addition of anti-PD-1 antibody to Lm AnxA2 vaccine treatment potentially increased the cure rate of PDAC in this preclinical model, further supporting the combination of vaccine-based therapies with immune checkpoint inhibitors as a strategy for pancreatic cancer treatment.

TABLE 1

| | Median | % |
|---|---|---|
| | Survival | Disease-free |
| Group (n = 11 per group) | (Days) | at 75 days |
| Empty Lm followed sequentially by (>) IgG | 51 | 9 |
| Lm-AnxA2 > IgG | 62 | 18 |
| Empty Lm > anti-PD-1 Antibody | 52 | 27 |
| Lm-AnxA2 > anti-PD-1 Antibody | 63 | 46 |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of treating or preventing cancer in a subject comprising:
administering a vaccine to said subject;
administering a programmed death 1 (PD-1) inhibitor, a PD-1 ligand (PD-L1) inhibitor, or a combination thereof to said subject,
thereby treating or preventing said cancer in said subject.

2. The method of claim 1, wherein said cancer comprises a gastrointestinal cancer.

3. The method of claim 2, wherein said cancer comprises pancreatic cancer.

4. The method of claim 3, wherein said pancreatic cancer comprises a pancreatic ductal adenocarcinoma (PDA).

5. The method of claim 1, wherein said vaccine comprises an allogeneic PDA tumor cell engineered to secrete granulocyte macrophage colony-stimulating factor (GM-CSF).

6. The method of claim 5, wherein said vaccine comprises GM-CSF-secreting PDA vaccine (GVAX).

7. The method of claim 1, wherein said PD-1 inhibitor or said PD-L1 inhibitor comprises an anti-PD-1 antibody or an anti-PD-L1 antibody.

8. The method of claim 2, wherein infiltration of CD8+ T lymphocytes, activated CD8+ T cells, and interferon gamma (IFNγ) producing CD8+ T cells into PDA tumor microenvironment (TME) is increased.

9. The method of claim 1, further comprising the administration of an immune modulating dose of cyclophosphamide to said subject.

10. The method of claim 9, wherein the immune modulating dose of cyclophosphamide is 100 mg/kg.

11. The method of claim 9, wherein said cyclophosphamide is administered intraperitoneally or orally.

12. The method of claim 9, wherein regulatory T cells (Tregs) and cytotoxic T lymphocyte antigen-4 (CTLA-4) expression on T cells is inhibited.

13. The method of claim 1, wherein subject survival time is increased compared to PD-1 monotherapy or GVAX monotherapy alone.

14. The method of claim 9, wherein the percentage of CD69+ CD8+ T cells among CD8+ lymphocytes infiltrating TME increases compared to cyclophosphamide and GVAX alone.

15. The method of claim 2, wherein a PDA tumor is reduced or inhibited.

16. The method of claim 1, wherein said subject is a human.

17. The method of claim 1, wherein said PD-1 inhibitor or said PD-L1 inhibitor is administered twice or more.

18. The method of claim 9, wherein said cyclophosphamide is administered twice or more.

19. (canceled)

20. (canceled)

21. (canceled)

22. The method of claim 1, wherein said vaccine comprises a Listeria monocytogenes (Lm)-based vaccine.

23. The method of claim 22, wherein said Listeria monocytogenes (Lm)-based vaccine expresses an Annexin A2 (ANXA2) antigen.

24. The method of claim 23, wherein the Listeria monocytogenes (Lm)-based vaccine expressing an Annexin A2 (ANXA2) antigen is administered prior to administration of the programmed death 1 (PD-1) inhibitor or the PD-1 ligand (PD-L1) inhibitor.

25. The method of claim 1, wherein the PD-1 inhibitor and PD-L1 inhibitor are administered sequentially.

26. The method of claim 1, wherein the PD-1 inhibitor, the PD-L1 inhibitor, or a combination thereof are administered prior to administration of additional immune modulators.

27. The method of claim 1, wherein the PD-1inhibitor, the PD-L1 inhibitor, or a combination thereof are administered prior to administration of a targeted therapy.

28. A composition for the treatment or prevention of cancer comprising a Listeria monocytogenes (Lm)-based vaccine that expresses an ANXA2 antigen.

29. The composition of claim 28, further comprising a programmed death 1 (PD-1) inhibitor or a PD-1 ligand (PD-L1) inhibitor.

30. The composition of claim 28, wherein the programmed death 1 (PD-1) inhibitor or a PD-1 ligand (PD-L1) inhibitor comprises an anti-PD-1 antibody or an anti-PD-L1 antibody.

31. A method of treating or preventing cancer in a subject comprising:
administering a vaccine to said subject;
administering an agent that alters immune suppressive signals to said subject,
thereby treating or preventing said cancer in said subject.

32. The method of claim 31 wherein the vaccine is selected from the group comprising a Listeria-based vaccines engineered to express cancer antigens, a vaccine that facilitates effector T cell infiltration into pancreatic tumors, a whole cell vaccine, a dendritic cell vaccine, or a combination thereof.

33. The method of claim 32 wherein the agent that alters immune suppressive signals to said subject comprises a programmed death 1 (PD-1) inhibitor, a PD-1 ligand (PD-L1) inhibitor, or a combination thereof.
